(19) United States
(12) Patent Application Publication
(10) Pub. No.: US 2019/0237161 A1
(43) Pub. Date: Aug. 1, 2019

(54) ERROR REMOVAL USING IMPROVED LIBRARY PREPARATION METHODS

(71) Applicant: GRAIL, Inc., Menlo Park, CA (US)

(72) Inventors: Ravi Vijaya Satya, San Bruno, CA (US); Sante Gnerre, Mountain View, CA (US); Nicholas Eattock, Hercules, CA (US); Lijuan Ji, Santa Clara, CA (US); Curtis Tom, San Francisco, CA (US)

(21) Appl. No.: 16/231,307

(22) Filed: Dec. 21, 2018

Related U.S. Application Data
(60) Provisional application No. 62/609,951, filed on Dec. 22, 2017.

(51) Int. Cl. G16B 35/10; G16B 25/20; G16B 30/00; C12Q 1/6886; C12Q 1/6855; C12Q 1/6881

(52) U.S. Cl. CPC G16B 35/10; G16B 25/20; C12Q 1/6881; C12Q 1/6886; C12Q 1/6855; G16B 30/00

(57) ABSTRACT

Methods for preparing sequencing libraries from a DNA-containing test sample, as well as methods for reducing the occurrence of edge errors prior to sequencing, are provided.

### CROSS-REFERENCE TO RELATED APPLICATIONS

Under 35 U.S.C. § 119(e), this application claims priority benefit of the filing date of U.S. Provisional Patent Application No. 62/609,951, filed on Dec. 22, 2017, the disclosure of which application is herein incorporated by reference in its entirety.

### FIELD OF THE INVENTION

The present invention relates to molecular biology techniques and methods for preparing sequencing libraries from a DNA-containing test sample, as well as methods for reducing the occurrence of edge errors prior to sequencing.

### BACKGROUND OF THE INVENTION

Analysis of circulating cell-free DNA (cfDNA) using next generation sequencing (NGS) is recognized as a valuable tool for detection and diagnosis of cancer. Identifying variants indicative of cancer using NGS often requires deep sequencing of circulating cfDNA from a patient test sample. Alternatively, many tumor-derived variants can also be identified using less expensive, lower depth, whole exome sequencing approaches. However, errors introduced during sample preparation and sequencing can make accurate identification of variants difficult.

Current protocols for preparing a sequencing library from cfDNA typically include the steps of end repair, 3′ end A-tailing, ligation of sequencing adapters, and polymerase chain reaction (PCR) amplification. However, this approach for preparing a cfDNA library may results in relatively high error rates near the ends of the cfDNA molecules, referred to as edge errors. Accordingly, there is a need in the art for improved methods for reducing errors near the ends of DNA molecules during library preparation.

### SUMMARY OF THE INVENTION

Aspects of the invention include methods for preparing a sequencing library from a test sample comprising a plurality of double-stranded DNA (dsDNA) molecules, the methods comprising: (a) obtaining a test sample comprising a plurality of dsDNA molecules, wherein the dsDNA molecules comprise one or more free single-stranded DNA (ssDNA) overhangs at one or both ends of the dsDNA molecules; (b) treating the dsDNA molecules to remove the free ssDNA overhangs, thereby generating a plurality of blunt ended dsDNA molecules; (c) modifying the blunt ended dsDNA molecules for adapter ligation; (d) ligating a plurality of dsDNA adapters to the plurality of blunt ended dsDNA molecules obtained from step (c) to generate a plurality of dsDNA adapter-molecule constructs; and (e) amplifying the dsDNA adapter-molecule constructs to generate a sequencing library. In some embodiments, treating the dsDNA molecules to remove the free ssDNA overhangs comprises an exonuclease pretreatment step, a DNA template repair pretreatment step, a heat inactivation step, or a combination thereof. In some embodiments, a method further comprises: (f) sequencing the sequencing library to obtain a plurality of sequence reads; and (g) detecting the presence or absence of cancer, determining cancer status, monitoring cancer progression and/or determining a cancer classification from the plurality of sequence reads.

In some embodiments, the dsDNA molecules are cell-free DNA (cfDNA) fragments. In some embodiments, the cfDNA fragments originate from healthy cells and from cancer cells. In some embodiments, the test sample is from whole blood, a blood fraction, plasma, serum, urine, fecal matter, saliva, a tissue biopsy, pleural fluid, pericardial fluid, cerebrospinal fluid (CSF), or peritoneal fluid. In some embodiments, the free single-stranded overhang comprises a free 5′-end. In some embodiments, the free single-stranded DNA overhang comprises a free 3′-end. In some embodiments, the exonuclease pretreatment step comprises a single strand DNA nuclease. In some embodiments, the single-strand DNA nuclease is mung bean nuclease. In some embodiments, the single-stranded DNA nuclease is exonuclease VII. In some embodiments, removal of the free single-stranded DNA using the single-strand DNA nuclease results in a plurality of blunt ended dsDNA molecules. In some embodiments, modification of the plurality of dsDNA fragments comprises end-repairing and A-tailing prior to ligation step (d). In some embodiments, the adapters further comprise a sample-specific index sequence. In some embodiments, the adapters further comprise a universal priming site. In some embodiments, the adapters further comprise one or more sequencing oligonucleotides for use in cluster generation and/or sequencing.

In some embodiments, the sequence reads are obtained from next-generation sequencing (NGS). In some embodiments, the sequence reads are obtained from massively parallel sequencing using sequencing-by-synthesis. In some embodiments, the sequence reads are obtained from paired-end sequencing.

In some embodiments, monitoring cancer progression further comprises monitoring disease progression, monitoring therapy, or monitoring cancer growth. In some embodiments, the cancer classification further comprises determining cancer type and/or cancer tissue of origin. In some embodiments, monitoring cancer progression further comprises monitoring disease progression, monitoring therapy, or monitoring cancer growth. In some embodiments, the cancer comprises a carcinoma, a sarcoma, a myeloma, a leukemia, a lymphoma, a blastoma, a germ cell tumor, or any combination thereof.

Aspects of the invention include methods for preparing a sequencing library from a test sample comprising a plurality of double-stranded DNA (dsDNA) molecules, the methods comprising: (a) obtaining a test sample comprising a plurality of dsDNA molecules; (b) treating the dsDNA molecules to remove and/or repair one or more uracil residues within the dsDNA molecules; (c) modifying the plurality of dsDNA fragments for adapter ligation; (d) ligating a plurality of dsDNA adapters to the plurality of dsDNA molecules obtained from step (c) to generate a plurality of dsDNA adapter-molecule constructs; and (e) amplifying the dsDNA adapter-molecule constructs to generate a sequencing library. In some embodiments, a method further comprises: (f) sequencing the sequencing library to obtain a plurality of sequence reads; and (g) detecting the presence or absence of cancer, determining cancer status, monitoring cancer progression and/or determining a cancer classification from the plurality of sequence reads.

In some embodiments, the dsDNA molecules are cell-free DNA (cfDNA) fragments. In some embodiments, the cfDNA fragments originate from healthy cells and from cancer cells. In some embodiments, the test sample is from whole blood, a blood fraction, plasma, serum, urine, fecal matter, saliva, a tissue biopsy, pleural fluid, pericardial fluid, cerebrospinal fluid (CSF), or peritoneal fluid. In some embodiments, a uracil-specific excision reagent is used to remove one or more uracil residues from the dsDNA molecules. In some embodiments, the removed uracil residue is replaced with a cytosine residue using a DNA polymerase and/or a DNA ligase. In some embodiments, the dsDNA molecules are further treated, prior to ligation step (d), to remove the free single-stranded overhangs, thereby generating a plurality of blunt ended dsDNA molecules. In some embodiments, further treating the dsDNA molecules to remove the free single-stranded overhangs comprises an exonuclease pretreatment step, a DNA template repair pretreatment step, a heat inactivation step, or a combination thereof.

In some embodiments, the free single-stranded DNA overhang comprises a free 5′-end. In some embodiments, the free single-stranded DNA overhang comprises a free 3′-end. In some embodiments, the exonuclease pretreatment step comprises a single strand DNA nuclease. In some embodiments, the single-strand DNA nuclease is mung bean nuclease. In some embodiments, the single-stranded DNA nuclease is exonuclease VII. In some embodiments, removal of the free single-stranded DNA using the single-strand DNA nuclease results in a plurality of blunt ended dsDNA molecules. In some embodiments, modification of the plurality of dsDNA fragments comprises end-repairing and A-tailing prior to ligation step (d). In some embodiments, the adapters further comprise a sample-specific index sequence. In some embodiments, the adapters further comprise a universal priming site. In some embodiments, the adapters further comprise one or more sequencing oligonucleotides for use in cluster generation and/or sequencing.

In some embodiments, the sequence reads are obtained from next-generation sequencing (NGS). In some embodiments, the sequence reads are obtained from massively parallel sequencing using sequencing-by-synthesis. In some embodiments, the sequence reads are obtained from paired-end sequencing.

In some embodiments, monitoring cancer progression further comprises monitoring disease progression, monitoring therapy, or monitoring cancer growth. In some embodiments, the cancer classification further comprises determining cancer type and/or cancer tissue of origin. In some embodiments, monitoring cancer progression further comprises monitoring disease progression, monitoring therapy, or monitoring cancer growth.

### BRIEF DESCRIPTION OF THE DRAWINGS

FIG. 1 is a plot showing the distribution of variants in sequencing reads from eleven cfDNA cancer patient samples;

FIG. 2A is a screenshot of the Integrative Genome Viewer (IGV) interface sequence alignment showing the G>A source variant on reads mapping to the forward strand;

FIG. 2B is a screenshot of the IGV interface sequence alignment showing C>T variants on reads mapping to the reverse strand;

FIG. 3 is a plot showing the number of reads supporting a single variant in the a first cfDNA sample set from blood plasma, a second cfDNA sample set from blood plasma, and a third sample set comprising genomic DNA from tissue samples;

FIG. 4 illustrates a schematic diagram of a process that may lead to edge errors during cfDNA library preparation;

FIG. 5 is a panel of various plots showing edge errors in sequencing reads from a cancer patient's cfDNA sample (cfDNA-mbc);

FIG. 6 is a panel of various plots showing edge errors in sequencing reads from a cancer patient's genomic DNA sample (gDNA-WBC-mbc);

FIG. 7 is a panel of various plots showing edge errors in sequencing reads from a healthy individual's cfDNA sample (cfDNA-healthy);

FIG. 8 is a panel of various plots showing G>A errors that occur in different trinucleotide contexts in sequencing reads from a cancer patient's cfDNA sample (cfDNA-mbc);

FIG. 9 is a panel of various plots showing C>T errors that occur in different trinucleotide contexts in sequencing reads from a cancer patient's cfDNA sample (cfDNA-mbc);

FIG. 10A is a plot showing 3′ G>A errors in the four different datasets;

FIG. 10B is a plot showing the G>A edge error rate for the cfDNA samples and the genomic DNA samples in the four datasets;

FIG. 11 is a flow diagram illustrating a method of reducing or substantially eliminating edge errors in a sequencing library using an enzymatic digestion step to remove the 3′ and/or 5′ overhanging ends of double-stranded DNA prior to ligation of sequencing adapters;

FIG. 12 is a flow diagram illustrating a method for detecting cancer, screening for cancer, determining cancer status, monitoring cancer progression, and/or determining a cancer classification, in accordance with the present invention.

FIG. 13 is a flow diagram illustrating a method of reducing or substantially eliminating uracil-induced edge errors in a sequencing library using a uracil excision and repair process prior to end repair and ligation of sequencing adapters; and

FIG. 14 is a flow diagram illustrating a method for detecting cancer, screening for cancer, determining cancer status, monitoring cancer progression, and/or determining a cancer classification, in accordance with the present invention.

FIG. 15 is graph showing percentage of duplex DNA as a function of various pretreatment protocols used to generate a sequencing library.

FIG. 16 is a graph showing read substitution error rate as a function of various pretreatment protocols used to generate a sequencing library.

FIG. 17 is a graph showing collapsed reads as a function of reaction mixture contents.

FIG. 18 is a graph showing normalized collapsed reads as a function of reaction mixture contents.

### DEFINITIONS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application, as do the following, each of which is incorporated by reference herein in its entirety: Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

All publications mentioned herein are expressly incorporated herein by reference to disclose and describe the methods and/or materials in connection with which the publications are cited.

The term “amplicon” as used herein means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26: 2150-2155 (1998), and like references.

As used herein, the term “amplifying” means performing an amplification reaction. A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger polynucleotide molecule. A polynucleotide, for example, can be broken up, or fragmented into, a plurality of segments, either through natural processes, as is the case with, e.g., cfDNA fragments that can naturally occur within a biological sample, or through in vitro manipulation. Various methods of fragmenting nucleic acids are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al, U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); the disclosures of which are hereby incorporated by reference herein in their entireties. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references, which are incorporated by reference herein in their entireties: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); and Becker-Andre et al, Nucleic Acids Research, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′-end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic acid amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2^{nd} Edition (Cold Spring Harbor Press, New York, 2003).

The terms “unique sequence tag”, “sequence tag”, “tag”, “unique molecular identifier”, “UMI”, or “barcode”, as used interchangeably herein, refer to an oligonucleotide that is attached to a polynucleotide or template molecule and is used to identify and/or track the polynucleotide or template in a reaction or a series of reactions. A sequence tag may be attached to the 3′- or 5′-end of a polynucleotide or template, or it may be inserted into the interior of such polynucleotide or template to form a linear conjugate, sometimes referred to herein as a “tagged polynucleotide,” or “tagged template,” or the like. Sequence tags may vary widely in size and compositions; the following references, which are incorporated herein by reference in their entireties, provide guidance for selecting sets of sequence tags appropriate for particular embodiments: Brenner, U.S. Pat. No. 5,635,400; Brenner and Macevicz, U.S. Pat. No. 7,537,897; Brenner et al, Proc. Natl. Acad. Sci., 97: 1665-1670 (2000); Church et al, European patent publication 0 303 459; Shoemaker et al, Nature Genetics, 14: 450-456 (1996); Morris et al, European patent publication 0799897A1; Wallace, U.S. Pat. No. 5,981,179; and the like. Lengths and compositions of sequence tags can vary widely, and the selection of particular lengths and/or compositions depends on several factors including, without limitation, how tags are used to generate a readout, e.g., via a hybridization reaction or via an enzymatic reaction, such as sequencing; whether they are labeled, e.g., with a fluorescent dye or the like; the number of distinguishable oligonucleotide tags required to unambiguously identify a set of polynucleotides, and the like, and how different the tags of a particular set must be in order to ensure reliable identification, e.g., freedom from cross hybridization or misidentification from sequencing errors. In one aspect, sequence tags can each have a length within a range of from about 2 to about 36 nucleotides, or from about 4 to about 30 nucleotides, or from about 4 to about 20 nucleotides, or from about 8 to about 20 nucleotides, or from about 6 to about 10 nucleotides. In one aspect, sets of sequence tags are used, wherein each sequence tag of a set has a unique nucleotide sequence that differs from that of every other tag of the same set by at least two bases; in another aspect, sets of sequence tags are used wherein the sequence of each tag of a set differs from that of every other tag of the same set by at least three bases.

The term “enrich” as used herein means to increase a proportion of one or more target nucleic acids in a sample. An “enriched” sample or sequencing library is therefore a sample or sequencing library in which a proportion of one of more target nucleic acids has been increased with respect to non-target nucleic acids in the sample.

The terms “subject” and “patient” are used interchangeably herein and refer to a human or non-human animal who is known to have, or potentially has, a medical condition or disorder, such as, e.g., a cancer.

The term “sequence read” as used herein refers to nucleotide sequences read from a sample obtained from a subject. Sequence reads can be obtained through various methods known in the art.

The terms “circulating tumor DNA” or “ctDNA” and “circulating tumor RNA” or “ctRNA” refer to nucleic acid fragments (DNA or RNA) that originate from tumor cells or other types of cancer cells, which may be released into a subject's bloodstream as a result of biological processes, such as apoptosis or necrosis of dying cells, or may be actively released by viable tumor cells.

### DETAILED DESCRIPTION OF THE INVENTION

The present invention is directed to a process for identifying an edge error source in sequencing reads, and methods of reducing the occurrence of edge errors prior to sequencing. The methods of the present invention are based, fully or in part, on the observation that cfDNA sequencing libraries prepared using a library preparation protocol that includes an end repair process have relatively high levels of errors mapping to the ends (e.g., the 3′ ends) of sequence reads. These “edge errors” are likely due to relatively high DNA damage rates (e.g., deamination of cytosine to uracil) in single-stranded regions of cfDNA fragments (e.g., 3′- or 5′-overhanging ends). In various embodiments, edge errors in sequencing libraries prepared from double-stranded DNA samples can be reduced or substantially eliminated using an improved library preparation protocol. Because the edge errors are reduced or substantially eliminated, sequence noise is reduced and sensitivity in variant calling is increased.

In one embodiment, the end repair process in a typical library preparation protocol is replaced with an enzymatic digestion step to remove the 3′ and/or 5′ overhanging ends of a double-stranded DNA molecule prior to ligation of sequencing adapters.

In another embodiment, a uracil excision and repair process is used to remove a uracil residue(s) from damaged DNA prior to end repair and ligation of sequencing adapters.

In still another embodiment, a computational approach is used to identify and correct edge errors in sequencing reads.

### Edge Errors

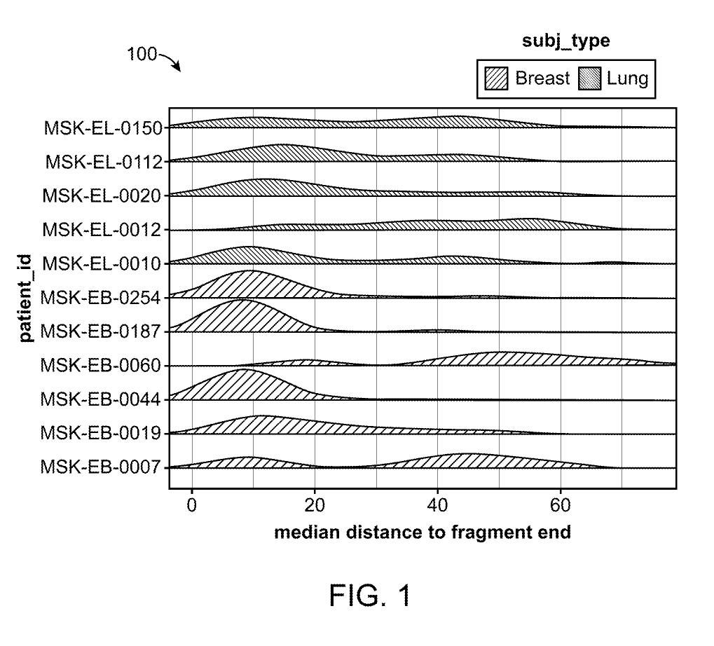

In the analysis of sequencing data from cfDNA patient samples, it was observed that some patient samples have a relatively high number of called variants with strand bias (i.e., reads supporting a variant were either from one strand or the other). Within the reads supporting these variants, the observed variants typically occur near the ends of the sequence reads. FIG. 1 is a plot 100 showing the distribution of variants in sequence reads from eleven cfDNA cancer patient samples. As demonstrated in FIG. 1, sequence variants are observed more frequently at the end of the sequence reads (e.g., as shown, typically within 20 bp of the ends of the reads).

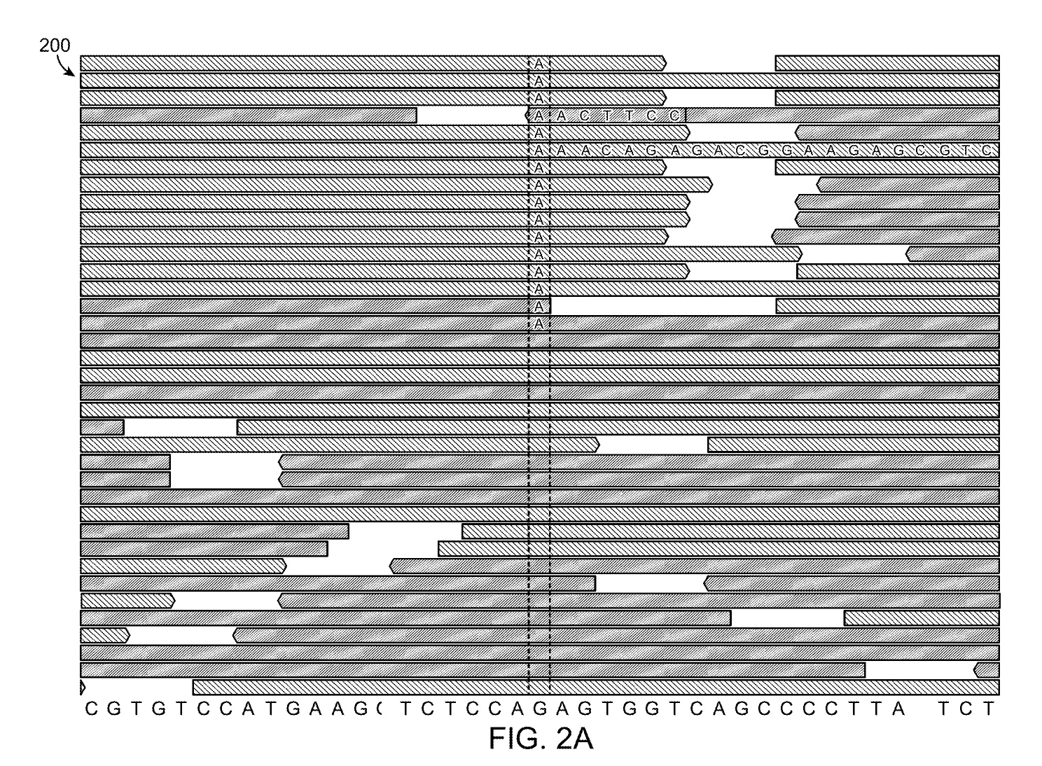

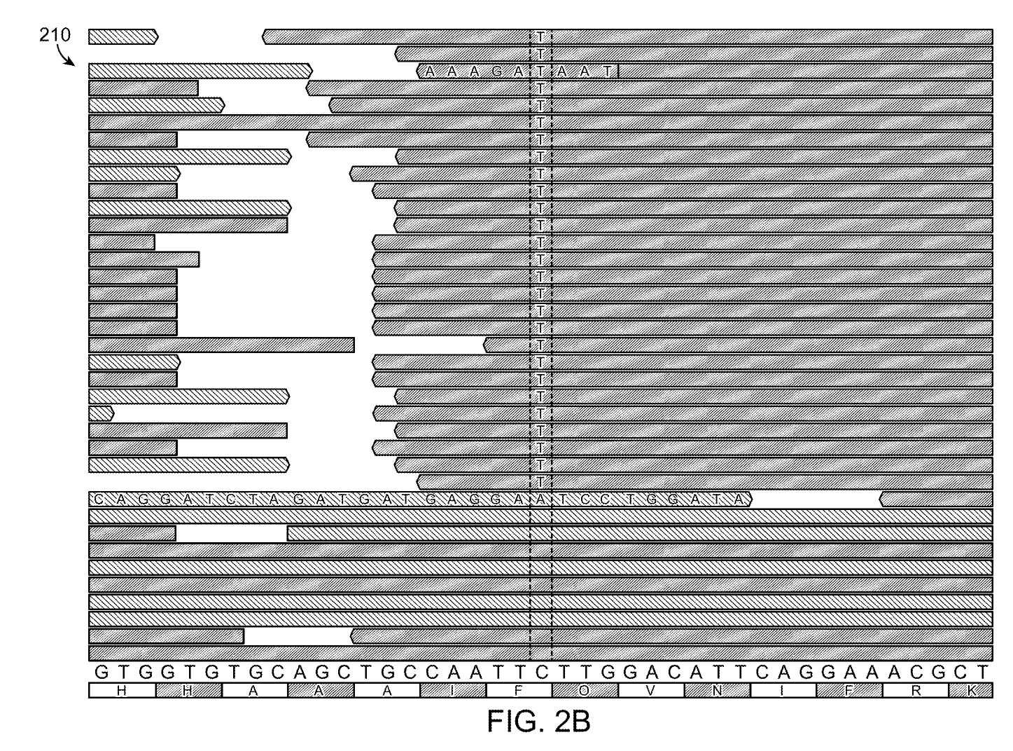

Further analysis of the dataset(s) identified the variant source as typically resulting in a change of G to A, i.e., G>A (on the read strand). FIG. 2A is a screenshot 200 of the Integrative Genome Viewer (IGV) interface sequence alignment showing an example of the G>A source variant on reads mapping to the forward strand. FIG. 2B is a screenshot 210 of the IGV interface sequence alignment showing an example of C>T variants on reads mapping to the reverse strand.

A majority of observed variants (about 90%) occur in non-duplex reads. Because the reads are non-duplex reads, all the reads are derived from only one of the original strands (i.e., either the forward or reverse strand, but not both). Typically, the trinucleotide context in which these errors occur is NGA (N={A, G, C, or T}), with TGA>TAA the most common sequence context (about 40% to about 50% occurrence) observed. Further, the observed errors occur predominantly at the 3′ end of the read (about 90% occurrence); however, the observed variants rarely occur at the 5′ end of reads. To gain insight into the cause of these edge-effect related variants, a first dataset (art_cfDNA (n=89 cfDNA samples from healthy individuals)), a second dataset (msk_cfDNA (n=357 cfDNA samples from cancer patients+healthy individuals)), and a third dataset (msk_gDNA (n=333 genomic DNA samples from cancer patients+healthy individuals)) were evaluated for a subset (n=270) of the edge-effect related variants.

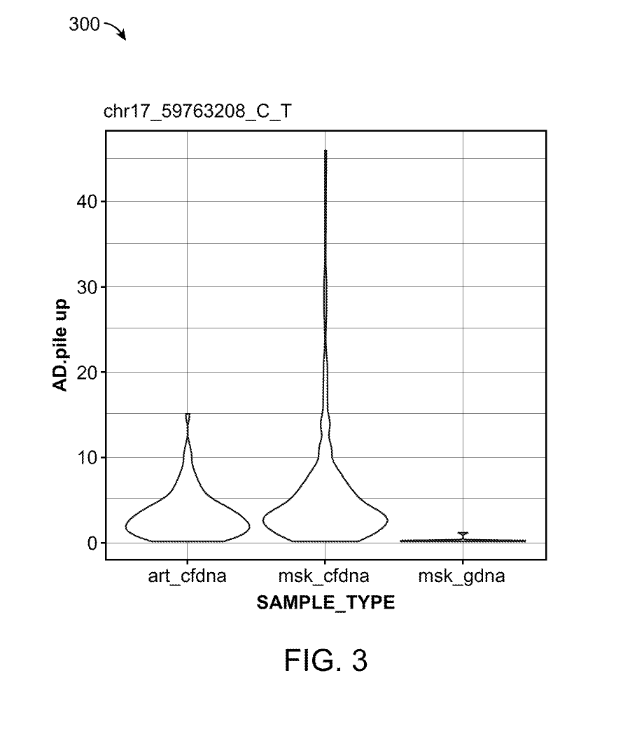

FIG. 3 is a plot 300 showing the number of reads supporting a single variant in three separate datasets (the first, second, and third datasets described above). Plot 300 is representative of about half of the variants observed in a dataset comprising 270-subset of edge-effect variants (data not shown). The data show that the edge-effect related variant occurs more frequently in reads from the cfDNA samples from healthy individuals (art_cfDNA) and healthy and cancer patients (msk_cfDNA; patient+healthy), than in reads from genomic DNA samples (msk_gDNA; patient+healthy).

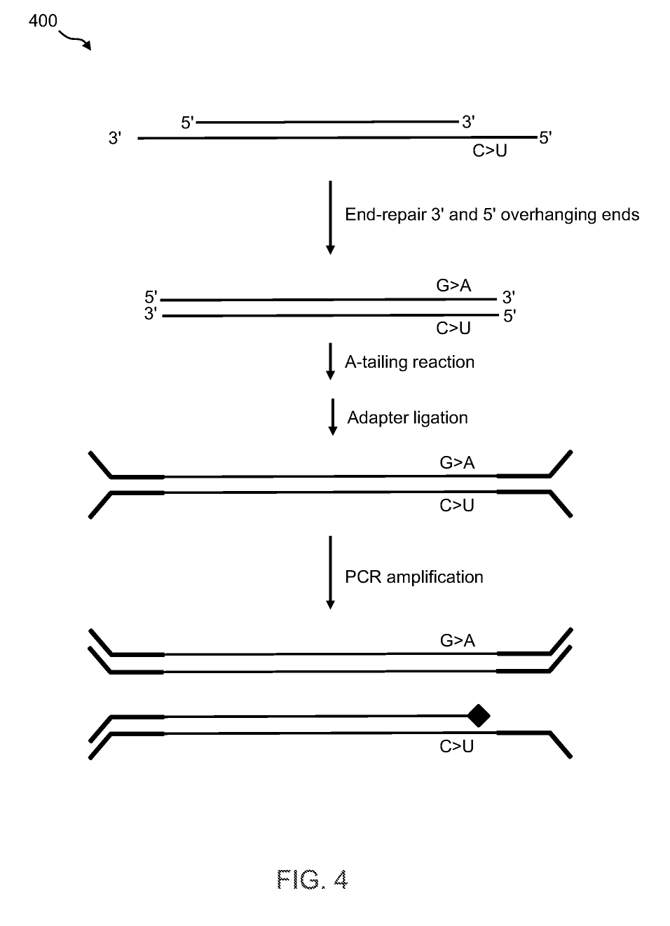

Based on the observation that the source variant is typically G>A (on reads mapping to the forward strand), occurs near the 3′ end of the read (rarely at the 5′ end of the read), and the underlying phenomenon occurs in cfDNA samples (not in genomic DNA samples) it was hypothesized that the end repair process used in a conventional library preparation protocol may result in the occurrence of these edge errors. In conventional library preparation processes, the end repair process uses DNA polymerase to fill in the complementary strand for 5′ overhangs and digests the 3′ overhangs generating a blunt-ended double-stranded fragment. Any errors within the 5′ overhang are copied into the newly synthesized complementary strand. FIG. 4 illustrates a schematic diagram of an example library preparation process 400 that results in observed edge errors in a cfDNA molecules. A double-stranded cfDNA molecule can include a 3′ overhanging end and a 5′ overhanging end. The single-stranded overhanging regions are susceptible to deamination of cytosine to uracil (indicated by C>U). In a conventional library preparation protocol, an end-repair reaction is performed using T4 DNA polymerase to digest the 3′ overhang and fill in the complementary strand for the 5′ overhang, generating a blunt-ended double-stranded cfDNA molecule. If a miscoding lesion, e.g., C>U, is present in the 5′ overhang (e.g., due to deamination), the DNA polymerase will insert a complementary adenine opposite the miscoding uracil and it will appear as a G>A mutation on the complementary strand. Other polymerase-mediated errors may also occur during this extension reaction (not shown). A-tailing and adapter ligation reactions are then performed to attach sequencing adapters to the blunt-ended double-stranded cfDNA. A PCR step is then performed to enrich for adapter ligated molecules. At this step, the end-repaired strand (i.e., the strand with the G>A substitution) is readily amplified. However, about 95% of the time, the polymerase used for PCR is blocked at the uracil base while copying the strand with the original 5′ overhang (i.e., the strand with the C>U deamination). Subsequently, either the nondamaged strand is sequenced and a complementary G to A substitution will be observed near the 3′-end of the sequence or, alternatively, the original damaged strand is sequenced, and a C to T substitution as a result of the uracil residue will be observed near the 5′-end of the sequence.

To test the hypothesis that the end repair process is responsible for edge errors, the base composition at the 3′ and 5′ ends of fragments was analyzed. If the hypothesis is correct, several observations were expected: 1) edge errors would occur in a high number of reads (i.e., not just limited to a few variant loci); 2) relatively high levels of G>A errors at the 3′ ends of reads and relatively lower levels of C>T errors at the 5′ ends of reads would be observed; 3) a higher error rate in other contexts near the 3′ end as compared to the 5′ end of reads would be observed; and 4) edge errors would be observed in reads from cfDNA samples from both healthy individuals and cancer patients.

All collapsed error-corrected reads in three separate datasets (the art_cfDNA, msk_cfDNA, and msk_gDNA datasets) were processed and all mismatches with the reference genome were enumerated (each error was counted only once). Any mismatch that could be a germline variant (i.e., greater than 5% allele fraction) was ignored. The distance of each error to the closest end (i.e., either the 3′ or 5′ end) was tabulated. The number of errors for A>C, A>G, A>T, C>A, C>G, C>T, G>A, G>C, G>T, T>A, T>C, and T>G substitutions in stitched non-duplex reads was plotted as a function of distance to the closest end of the read (i.e., 3′ or 5′ end of the read).

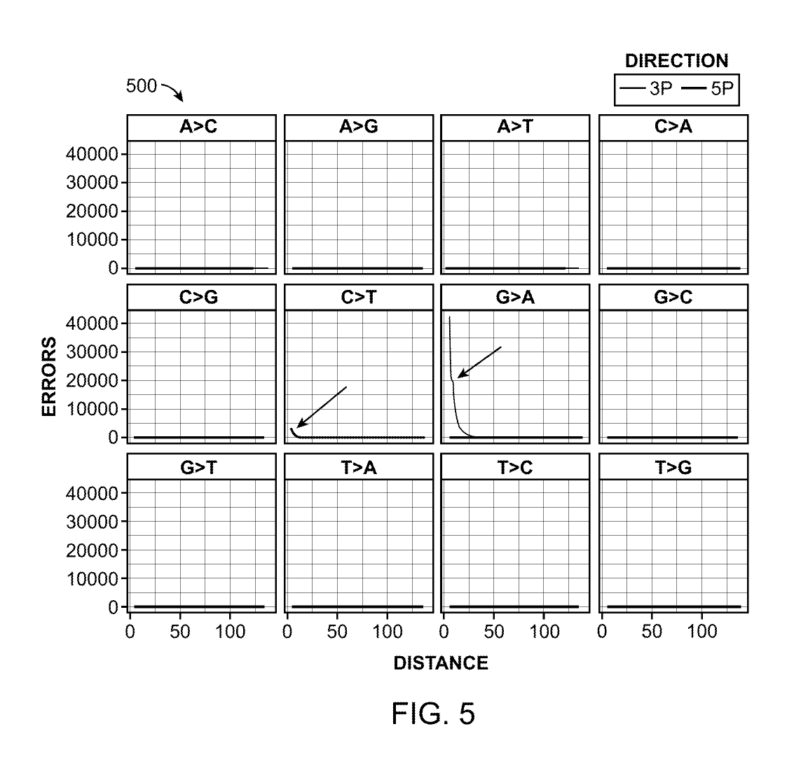

FIG. 5 shows a panel 500 of various plots showing the number of edge errors observed in sequence reads from a cfDNA sample obtained from a subject with cancer (cfDNA-mbc, obtained from a metastatic breast cancer patient). The red line represents the number of errors that map closer to the 3′ end of a sequence read and the green line represents the number of errors that map closer to the 5′ end of a sequence read. The data show that there is a relatively large number of G>A errors observed near the 3′ end of the sequence reads (red line). There are also a relatively small number of C>T errors observed near the 5′ end of the sequence reads (green line). Other errors, e.g., C>A, are also present, but at much lower levels compared to G>A errors.

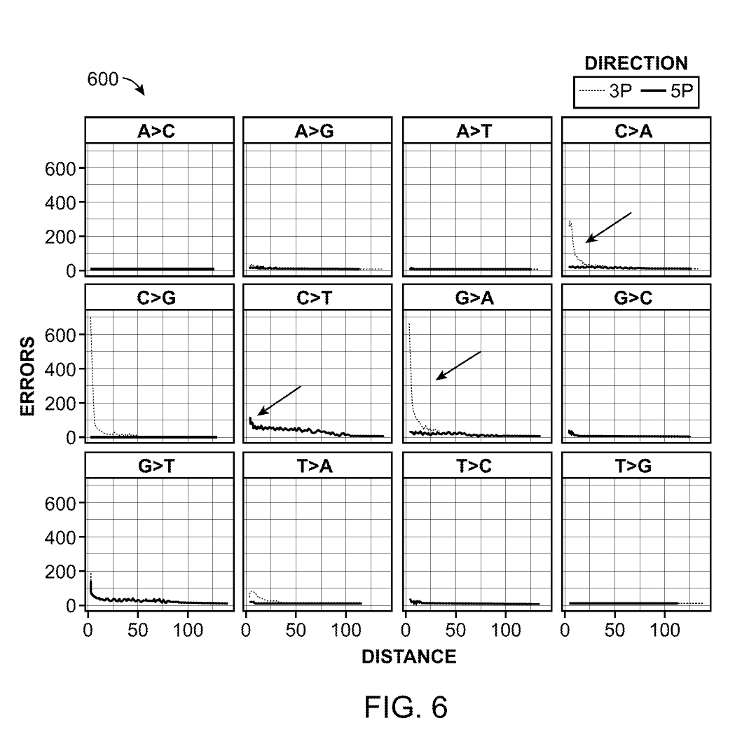

FIG. 6 shows a panel 600 of various plots showing the number of edge errors observed in sequence reads from a genomic DNA sample obtained from a subject with cancer (gDNA-WBC-mbc), sequence reads from WBCs obtained from a metastatic breast cancer patient). The genomic DNA sample is from the same cancer patient as the cfDNA sample of FIG. 5. The data show that G>A, C>T, and other edge errors also occur in the sequence reads from the genomic DNA sample, but at much lower rates (i.e., about 2 orders of magnitude lower) compared to the number of errors observed in the cfDNA sample from the same subject. For example, the number of G>A substitutions observed near the 3′ end of reads is about 40,000 in sequence reads from the cfDNA sample (FIG. 5) compared to about 600 G>A substitutions in sequence reads from the genomic DNA sample (FIG. 6).

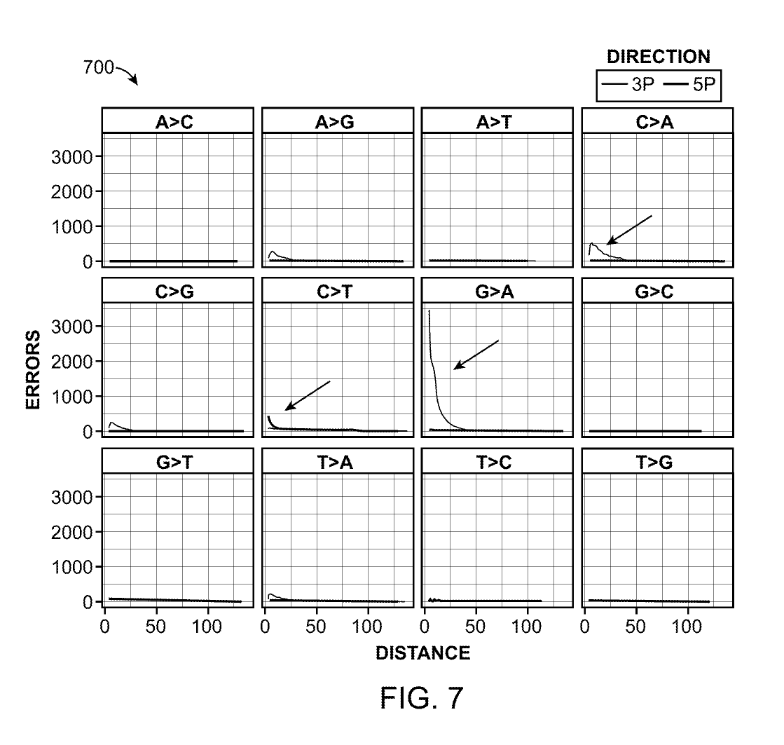

FIG. 7 shows a panel 700 of various plots showing the number of edge errors in sequence reads from a cfDNA sample obtained from a healthy subject (cfDNA-healthy). The data show that G>A, C>T, and other edge errors also occur in the sequence reads from the cfDNA sample from a healthy subject, but at much lower rates compared the number of errors observed in the reads from the cancer patient cfDNA sample of FIG. 5.

As shown in FIGS. 5, 6 and 7, the degree to which these edge errors occur varies from sample to sample. The variation in the degree of edge error occurrence may be due, for example, to pre-analytical factors (e.g., sample storage conditions) and/or biological factors (e.g., circulating tumor in cancer samples). Some of the edge errors have a relatively high number (e.g., 30 to 40 fragments with the same error at the same location) of reads supporting a single variant (data not shown). Edge errors with a relatively high number of supporting reads may be due, for example, to cytosine deamination occurring in specific sites/contexts.

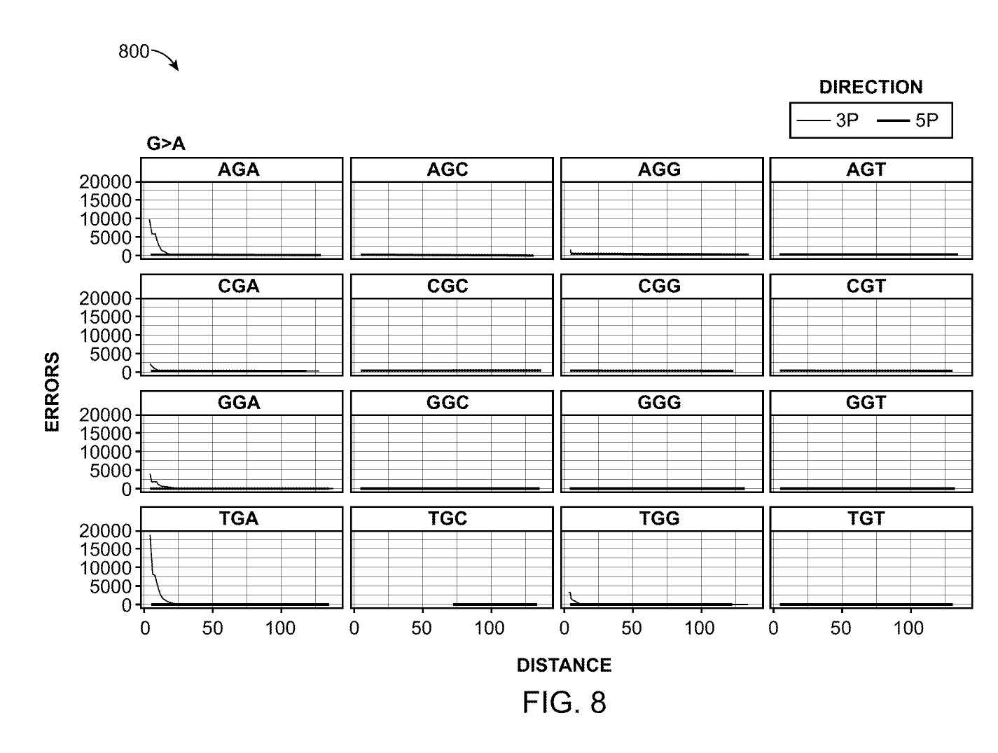

FIG. 8 shows a panel 800 of various plots showing the number of G>A errors that occur in different trinucleotide contexts in sequence reads from the cfDNA sample obtained from a subject with cancer (cfDNA-mbc). The data show that about half the time G>A errors mapping to the 3′ end of reads occur within a sequence context of TGA (i.e., TGA>TAA).

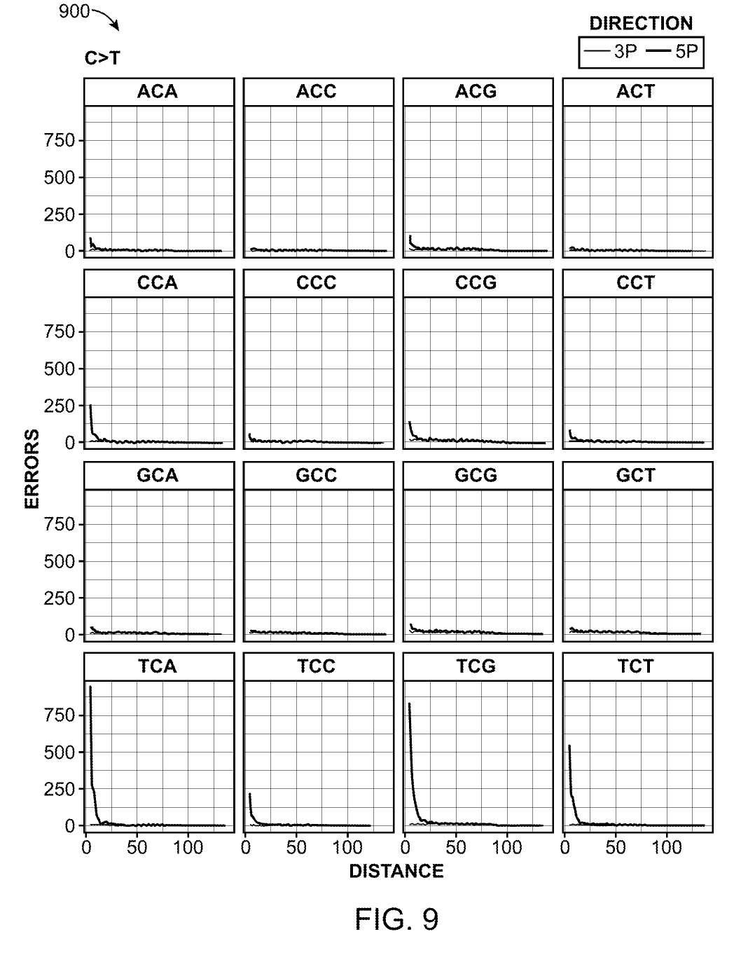

FIG. 9 shows a panel 900 of various plots showing the number of C>T errors that occur in different trinucleotide contexts in sequence reads from the cfDNA sample obtained from a subject with cancer (cfDNA-mbc). The data show that a relatively high number of C>T errors at the 5′ end of the reads occur within a sequence context of TCA (i.e., TCA>TTA) and TCG (TCG>TTG). The TCA context corresponds with the complementary context of TGA for G>A errors described with reference to FIG. 8. However, the TCG context for C>T errors does not correspond to the complementary context of CGA for G>A errors described with reference to FIG. 8 (i.e., the number of G>A errors in the CGA context is relatively low). This lack of complementarity in trinucleotide contexts may be due, for example, to variable polymerase blockage at the uracil base in different trinucleotide contexts.

To evaluate the degree of edge errors in sequencing reads from cfDNA and genomic DNA samples, four datasets were used: (1) cfDNA samples from healthy individuals (art; n=89); (2) cfDNA samples from healthy individuals (CCGA; n=150); (3) genomic DNA samples from cancer patients (merlin; n=215 cfDNA+152); and (4) genomic DNA samples from cancer patients (msk_techval; n=193 cfDNA+163).

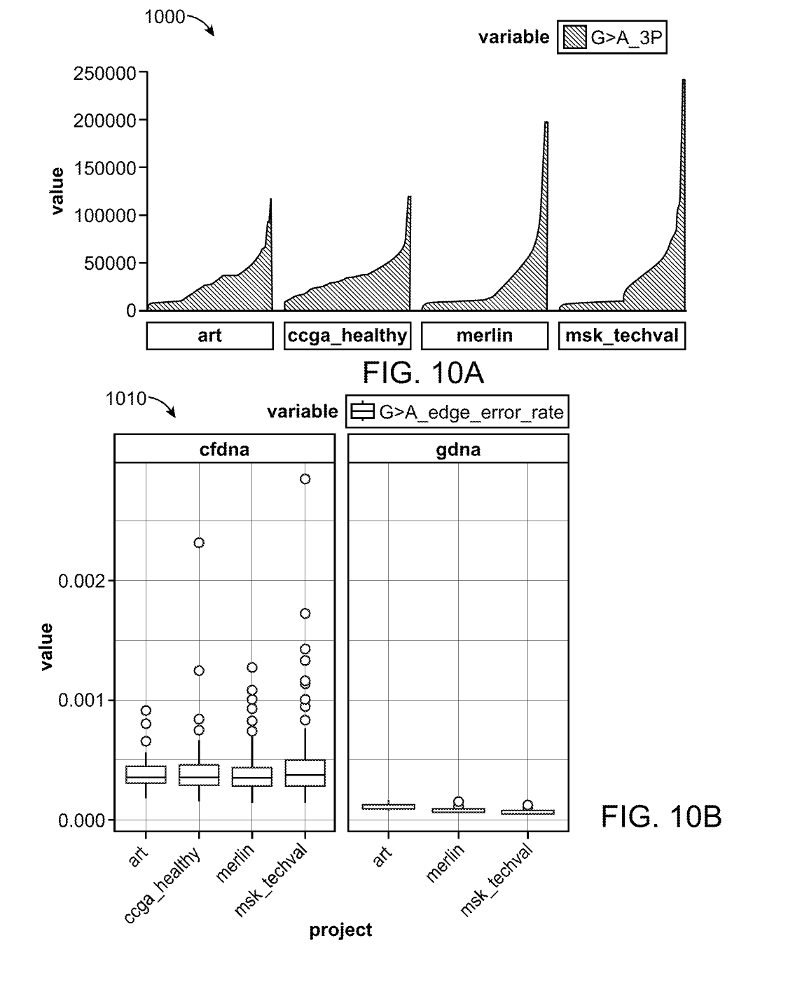

FIG. 10A is a plot 1000 showing the number of 3′ G>A errors in datasets (1)-(4). The data show that some samples have a relatively high number of 3′ G>A errors. The relatively flat areas in the merlin and msk_techval plots are representative of the genomic DNA samples in the dataset. In cfDNA samples, up to 50% of total errors observed are believed to result from error occurring in the 5′ overhangs

FIG. 10B is a plot 1010 showing the G>A edge error rate for the cfDNA samples and the genomic DNA samples in the four datasets. The error rate is the number of errors/total number of collapsed reads. The data show that G>A errors occur at a very low rate in the genomic DNA samples. The data also show that the G>A error rate is about the same in the cfDNA samples (i.e., samples from cancer patients and healthy individuals), with the exception of a few outliers. The data indicate that G>A edge errors may result from pre-analytical factors (e.g., sample storage conditions) rather than biological factors (e.g., cancer versus healthy individual). The error rate may be an underestimate because only stitched reads are considered and errors are knocked out from duplex reads (i.e., only non-duplex reads are considered).

Another approach that can be used to test the hypothesis that the end repair process is responsible for edge errors is to perform the PCR step of the library preparation protocol using a polymerase that can read through uracil residues (e.g., KAPA U+). In this approach, the original strand that includes the C>U deamination in the 5′ overhang is amplified by the polymerase and will be incorporated in the final sequencing library. If the hypothesis is correct, sequencing of both strands will show duplex errors with a balance between 3′ end G>A errors and 5′ end C>T errors.

### Error Removal Using Improved Library Preparation Protocols

Edge errors are due to relatively high DNA damage rates (e.g., deamination of cytosine to uracil) in single-stranded regions of DNA fragments (e.g., 5′ overhanging ends). In various embodiments, edge errors in sequencing libraries prepared from double-stranded DNA samples can be reduced or substantially eliminated using an improved library preparation protocol. Because the edge errors are reduced (e.g., about a 50% reduction in errors) or substantially eliminated, sequence noise is reduced and sensitivity in variant calling is increased.

In one embodiment, the end repair process in a typical library preparation protocol is replaced with an enzymatic digestion step to remove the 3′ and/or 5′ overhanging ends of a double-stranded DNA molecule prior to ligation of sequencing adapters. Because the single-stranded 3′ and/or 5′ overhanging ends are removed prior to subsequent processing steps, the incorporation of errors due, for example, to deamination of cytosine to uracil in the single-stranded regions of the cfDNA molecules is reduced or substantially eliminated.

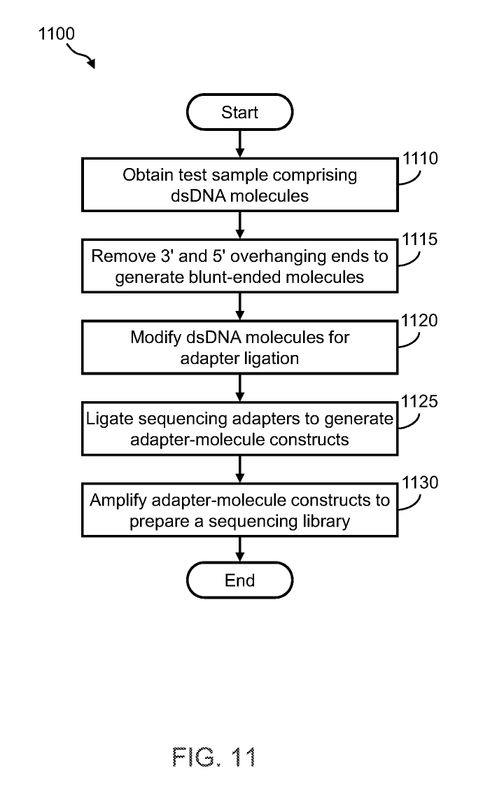

FIG. 11 is a flow diagram illustrating a method 1100 of reducing or substantially eliminating edge errors in a sequencing library using an enzymatic digestion step to remove the 3′ and/or 5′ overhanging ends of double-stranded DNA, in accordance with one embodiment of the present invention. Method 1100 includes, but is not limited to, the following steps.

At a step 1110, a DNA test sample is obtained from a subject (e.g., a patient). In one embodiment, the test sample may be a biological test sample selected from the group consisting of blood, plasma, serum, urine, saliva, fecal matter, and any combination thereof. Alternatively, the test sample or biological test sample may comprise a test sample selected from the group consisting of whole blood, a blood fraction, a tissue biopsy, pleural fluid, pericardial fluid, cerebrospinal fluid (CSF), and peritoneal fluid. In other embodiments, the sample is a plasma sample from a cancer patient, or a patient suspected of having cancer. In accordance with some embodiments, the test sample or biological test sample comprises a plurality of cell-free nucleic acids (e.g., cell-free DNA (cfDNA) and/or cell-free RNA (cfRNA)) fragments. In other embodiments, the test sample or biological test sample comprises a plurality of cell-free nucleic acids (e.g., cell-free DNA and RNA) fragments originating from healthy cells and from cancer cells. Optionally, in one embodiment, cell-free nucleic acids (e.g., cfDNA and/or cfRNA) can be extracted and/or purified from the test sample before proceeding with subsequent library preparation steps. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (Qiagen). In accordance with one embodiment, the DNA sample is a cfDNA sample from a cancer patient that includes double-stranded DNA molecules with single-stranded 3′ and/or 5′ overhanging ends.

At step 1115, the 3′ and/or 5′ single-stranded overhanging ends are removed using an enzymatic digestion reaction. In one embodiment, the 3′ and/or 5′ overhanging are removed using a single-stranded DNA nuclease to digest the single-stranded ends of the dsDNA molecules. In general, any known single-stranded DNA nuclease known in the art can be used for this step. For example, in one embodiment, the 3′ and/or 5′ overhanging ends are removed in a digestion reaction using mung bean nuclease (New England BioLabs, Ipswich, Mass.) to generating blunt-ended double-stranded dsDNA molecules. In another embodiment, the 3′ and/or 5′ overhanging ends are removed in a digestion reaction using exonuclease VII (New England BioLabs, Ipswich, Mass.) to generating blunt-ended double-stranded dsDNA molecules.

At step 1120, the double-stranded nucleic acid molecules are modified for adapter ligation. For example, the ends of dsDNA molecules are repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At step 1125 double-strand DNA adapters are ligated to the ends of the dsDNA molecules obtained from step 1120 to generate a plurality of dsDNA adapter-fragment constructs. The ligation reaction can be performed using any suitable ligation step (e.g., using a ligase) which joins the dsDNA adapters to the dsDNA fragments to form dsDNA adapter-fragment constructs. In one example, the ligation reaction is performed using T4 DNA ligase. In another example, T7 DNA ligase is used for adapter ligation to the modified nucleic acid molecules.

In one embodiment, the sequencing adapters can include a unique molecular identifier (UMI) sequence, such that, after library preparation, the sequencing library will include UMI tagged amplicons derived from dsDNA fragments. In one embodiment, unique sequence tags (e.g., unique molecular identifiers (UMIs)) can be used to identify unique nucleic acid sequences from a test sample. For example, differing unique sequence tags (UMIs) can be used to differentiate various unique nucleic acid sequence fragments originating from the test sample. In another embodiment, unique sequence tags (UMIs) can be used to reduce amplification bias, which is the asymmetric amplification of different targets due to differences in nucleic acid composition (e.g., high GC content). The unique sequence tags (UMIs) can also be used to discriminate between nucleic acid mutations that arise during amplification. In one embodiment, the unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag may comprise a short oligonucleotide sequence greater than about 5, 6, 7, 8, 9, 10, 11, 12, 13, 14, 15, 16, 17, or 18 nucleotides (nt) in length.

The unique sequence tags can be present in a multi-functional nucleic acid sequencing adapter, which sequencing adapter can comprise a unique sequence tag and/or a universal priming site. In another embodiment, the sequencing adapters utilized may include a universal primer and/or one or more sequencing oligonucleotides for use in subsequent cluster generation and/or sequencing (e.g., known P5 and P7 sequences for used in sequencing by synthesis (SBS) (Illumina, San Diego, Calif.)).

At step 1130, the dsDNA adapter-fragment constructs are amplified to generate a sequencing library. For example, the adapter-modified dsDNA molecules can be amplified by PCR using a DNA polymerase and a reaction mixture containing primers.

In another embodiment, a uracil excision and repair process is used to remove a uracil residue(s) from damaged DNA prior to end repair and ligation of sequencing adapters. Because a uracil residue(s) is removed prior to subsequent processing steps, the incorporation of errors due, for example, to deamination of cytosine to uracil in the single-stranded regions of double-stranded DNA molecules is reduced or substantially eliminated.

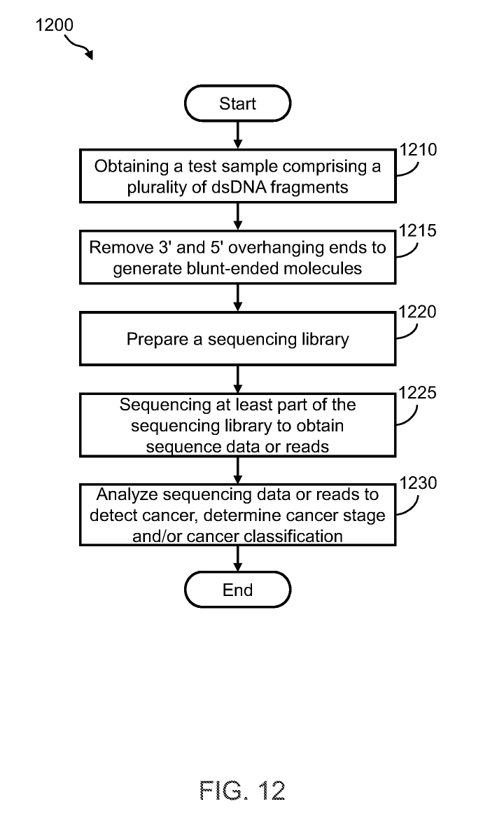

FIG. 12 is a flow diagram illustrating a method 1200 for preparing a sequencing library from a cell-free DNA test sample for use thereof in detecting cancer, determining cancer status, monitoring cancer progression, and/or determining a cancer classification. As shown in FIG. 12, at step 1210, a test sample (e.g., a biological test sample) is obtained from a subject (e.g., a patient). As noted above, the test sample may be a biological test sample selected from the group consisting of blood, whole blood, a blood fraction, plasma, serum, urine, fecal matter, saliva, a tissue biopsy, pleural fluid, pericardial fluid, cerebrospinal fluid (CSF), peritoneal fluid sample, and any combination thereof. In certain aspects, the test sample or biological test sample comprises a plurality of cell-free nucleic acids (e.g., cell-free DNA (cfDNA) and/or cell-free RNA (cfRNA)) fragments. In certain embodiments, the test sample or biological test sample comprises a plurality of cell-free nucleic acids (e.g., cell-free DNA and RNA) fragments originating from healthy cells and from cancer cells. In one embodiment, cell-free nucleic acids (e.g., cfDNA and/or cfRNA) can be extracted and/or purified from the test sample before proceeding with subsequent library preparation steps.

At step 1215, the 3′ and/or 5′ single-stranded overhanging ends are removed using an enzymatic digestion reaction. In one embodiment, the 3′ and/or 5′ overhanging are removed using a single-stranded DNA nuclease to digest the single-stranded ends of the dsDNA molecules. In general, any known single-stranded DNA nuclease known in the art can be used for this step. For example, in one embodiment, the 3′ and/or 5′ overhanging ends are removed in a digestion reaction using mung bean nuclease (New England BioLabs, Ipswich, Mass.) to generating blunt-ended double-stranded dsDNA molecules.

At step 1220, double-strand DNA adapters are ligated to the dsDNA molecules obtained from step 1215 in a ligation reaction to generate a plurality of dsDNA adapter-molecule constructs. The ligation reaction can be performed using any suitable ligation step (e.g., using a ligase) which joins the dsDNA adapters to the dsDNA molecules to form dsDNA adapter-molecule constructs. In one example, the ligation reaction is performed using T4 DNA ligase. In another example, T7 DNA ligase is used for adapter ligation to the modified nucleic acid molecule. As described above, the ends of the dsDNA molecules may be repaired, phosphorylated and/or end-tailed prior to ligation of adapters to the ends of the dsDNA molecules.

As noted above, the dsDNA adapters may comprise a unique molecular identifier (UMI) sequence. The unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag may comprise a short oligonucleotide sequence greater than about 5, 6, 7, 8, 9, 10, 11, 12, 13, 14, 15, 16, 17, or 18 nucleotides (nt) in length. Also like the ssDNA adapters described above, the dsDNA adapters may include a universal primer and/or one or more sequencing oligonucleotides for use in subsequent cluster generation and/or sequencing (e.g., known P5 and P7 sequences for used in sequencing by synthesis (SBS) (Illumina, San Diego, Calif.)).

At step 1225 a portion of the sequence library is sequenced to obtain sequencing data or sequence reads, and the sequencing data or sequence reads analyzed. In general, any method known in the art can be used to obtain sequence data or sequence reads from a test sample. For example, in one embodiment, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step is performed prior to sequencing. In certain embodiments, the sequencing comprises whole genome sequencing (or shotgun sequencing) of the cfDNA library to provide sequence data or sequencing reads representative of a whole genome. In other embodiments, the sequencing comprises targeted sequencing of the cfDNA library. For example, the sequencing library can be enriched for specific target sequences (e.g., using a plurality of hybridization probes to pull down cfDNA fragments known to be, or suspected of being, indicative of cancer) and the targeted sequences sequenced.

At step 1230, the sequencing data or sequence reads can be analyzed for detecting the presence of absence of cancer, screening for cancer, determining cancer stage or status, monitoring cancer progression, and/or for determining a cancer classification (e.g., cancer type or cancer tissue of origin). In another embodiment, the sequencing data or sequence reads can be used to infer the presence or absence of cancer, cancer status and/or a cancer classification. For example, the sequencing data or sequencing reads can be analyzed to identify one or more mutational signatures indicative of cancer (see, e.g., U.S. Patent Application No. 62/469,984, filed Mar. 10, 2017). Alternatively, machine learning can be used for the detection and/or classification of cancer based on one or more parameters determined from sequencing data or sequencing reads (see, e.g., U.S. Patent Application No. 62/553,670, filed Sep. 1, 2017).

In one embodiment, the sequencing data or sequence reads can be analyzed to detect the presence or absence of, screening for, determine the stage or status of, monitor progression of, and/or classify a carcinoma, a sarcoma, a myeloma, a leukemia, a lymphoma, a blastoma, a germ cell tumor, or any combination thereof. In some embodiments, the carcinoma may be an adenocarcinoma. In other embodiments, the carcinoma may be a squamous cell carcinoma. In still other embodiments, the carcinoma is selected from the group consisting of: small cell lung cancer, non-small-cell lung, nasopharyngeal, colorectal, anal, liver, urinary bladder, cervical, testicular, ovarian, gastric, esophageal, head-and-neck, pancreatic, prostate, renal, thyroid, melanoma, and breast carcinoma. In another embodiment, the sequencing data or sequence reads can be analyzed to detect presence or absence of, screening for, determine the stage or status of, monitor progression of, and/or classify a sarcoma. In certain embodiments, the sarcoma can be selected from the group consisting of: osteosarcoma, chondrosarcoma, leiomyosarcoma, rhabdomyosarcoma, mesothelial sarcoma (mesothelioma), fibrosarcoma, angiosarcoma, liposarcoma, glioma, and astrocytoma. In still another embodiment, the sequencing data or sequence reads can be analyzed to detect presence or absence of, screening for, determine the stage or status of, monitor progression of, and/or classify leukemia. In certain embodiments, the leukemia can be selected from the group consisting of: myelogenous, granulocytic, lymphatic, lymphocytic, and lymphoblastic leukemia. In still another embodiment, the sequencing data or sequence reads can be used to detect presence or absence of, screening for, determine the stage or status of, monitor progression of, and/or classify a lymphoma. In certain embodiments, the lymphoma can be selected from the group consisting of: Hodgkin's lymphoma and Non-Hodgkin's lymphoma.

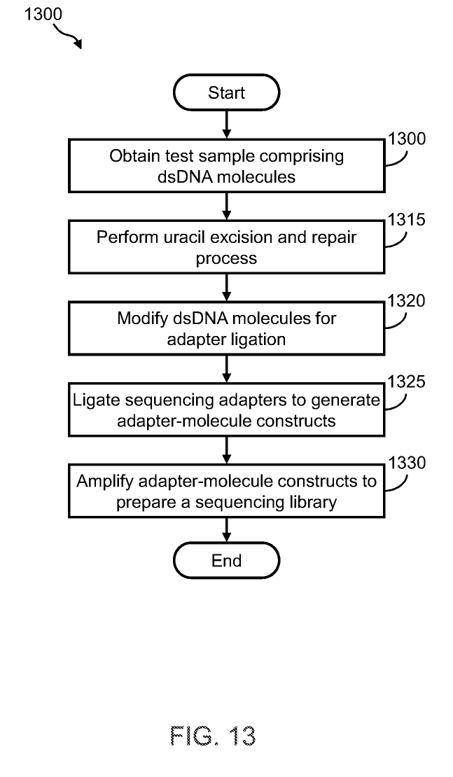

FIG. 13 illustrates a flow diagram of an example of a method 1300 of reducing or substantially eliminating uracil-induced edge errors in a sequencing library using a uracil excision and repair process prior to end repair and ligation of sequencing adapters. Method 1300 includes, but is not limited to, the following steps.

At a step 1310, a DNA sample is obtained. In one example, the DNA sample is a cfDNA sample from a cancer patient that includes double-stranded DNA molecules with single-stranded 3′ and/or 5′ overhanging ends.

At a step 1315, a uracil excision and repair process is performed. In one embodiment, a uracil-specific excision reagent (e.g., USER® Enzyme, available from New England Biolabs, Ipswich, Mass.) is used to remove a uracil residue(s) and the gap formed by excision of the residue(s) is filled by DNA polymerase and sealed by DNA ligase. In another embodiment, PreCR® Repair Mix (available from New England Biolabs, Ipswich, Mass.) is used to repair the damaged DNA prior to subsequent processing steps.

At a step 1320, the double-stranded nucleic acid molecules are modified for adapter ligation. For example, the ends of dsDNA molecules are repaired using, for example, T4 DNA polymerase and/or Klenow polymerase and phosphorylated with a polynucleotide kinase enzyme prior to ligation of the adapters. A single “A” deoxynucleotide is then added to the 3′ ends of dsDNA molecules using, for example, Taq polymerase enzyme, producing a single base 3′ overhang that is complementary to a 3′ base (e.g., a T) overhang on the dsDNA adapter.

At a step 1325, double-strand DNA adapters are ligated to the ends of the dsDNA molecules obtained from step 1310 to generate a plurality of dsDNA adapter-fragment constructs. The ligation reaction can be performed using any suitable ligation step (e.g., using a ligase) which joins the dsDNA adapters to the dsDNA fragments to form dsDNA adapter-fragment constructs. In one example, the ligation reaction is performed using T4 DNA ligase. In another example, T7 DNA ligase is used for adapter ligation to the modified nucleic acid molecules.

In one embodiment, the sequencing adapters can include a unique molecular identifier (UMI) sequence, such that, after library preparation, the sequencing library will include UMI tagged amplicons derived from dsDNA fragments. In one embodiment, unique sequence tags (e.g., unique molecular identifiers (UMIs)) can be used to identify unique nucleic acid sequences from a test sample. For example, differing unique sequence tags (UMIs) can be used to differentiate various unique nucleic acid sequence fragments originating from the test sample. In another embodiment, unique sequence tags (UMIs) can be used to reduce amplification bias, which is the asymmetric amplification of different targets due to differences in nucleic acid composition (e.g., high GC content). The unique sequence tags (UMIs) can also be used to discriminate between nucleic acid mutations that arise during amplification. In one embodiment, the unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag may comprise a short oligonucleotide sequence greater than about 5, 6, 7, 8, 9, 10, 11, 12, 13, 14, 15, 16, 17, or 18 nucleotides (nt) in length.

The unique sequence tags can be present in a multi-functional nucleic acid sequencing adapter, which sequencing adapter can comprise a unique sequence tag and/or a universal priming site. In another embodiment, the sequencing adapters utilized may include a universal primer and/or one or more sequencing oligonucleotides for use in subsequent cluster generation and/or sequencing (e.g., known P5 and P7 sequences for used in sequencing by synthesis (SBS) (Illumina, San Diego, Calif.)).

At step 1330, the dsDNA adapter-molecule constructs are amplified to generate a sequencing library. For example, the dsDN adapter-molecule constructs can be amplified by PCR using a DNA polymerase and a reaction mixture containing primers.

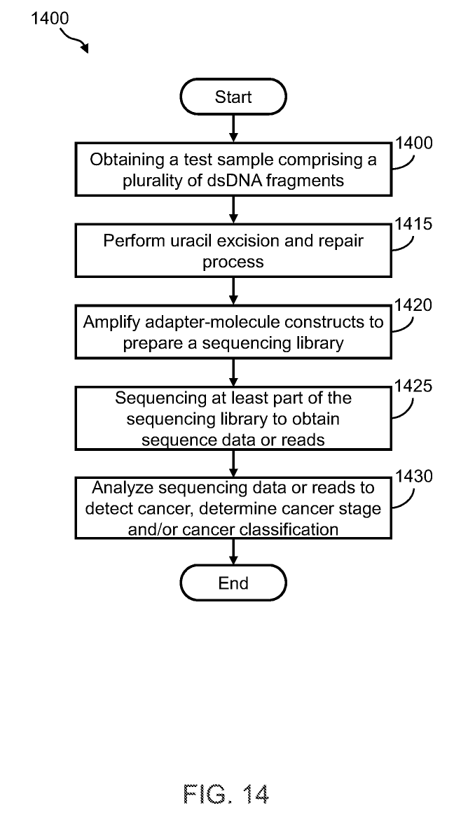

FIG. 14 is a flow diagram illustrating a method 1400 for preparing an enriched sequencing library from a cell-free DNA test sample for use thereof in detecting cancer, determining cancer status, monitoring cancer progression, and/or determining a cancer classification. As shown in FIG. 14, at step 1410, a test sample (e.g., a biological test sample) is obtained from a subject (e.g., a patient). As noted above, the test sample may be a biological test sample selected from the group consisting of blood, whole blood, a blood fraction, plasma, serum, urine, fecal matter, saliva, a tissue biopsy, pleural fluid, pericardial fluid, cerebrospinal fluid (CSF), peritoneal fluid sample, and any combination thereof. In certain aspects, the test sample or biological test sample comprises a plurality of cell-free nucleic acids (e.g., cell-free DNA (cfDNA) and/or cell-free RNA (cfRNA)) fragments. In certain embodiments, the test sample or biological test sample comprises a plurality of cell-free nucleic acids (e.g., cell-free DNA and RNA) fragments originating from healthy cells and from cancer cells. In one embodiment, cell-free nucleic acids (e.g., cfDNA and/or cfRNA) can be extracted and/or purified from the test sample before proceeding with subsequent library preparation steps.

At step 1415 a uracil excision and repair process is performed. In one embodiment, a uracil-specific excision reagent (e.g., USER® Enzyme, available from New England Biolabs, Ipswich, Mass.) is used to remove a uracil residue(s) and the gap formed by excision of the residue(s) is filled by DNA polymerase and sealed by DNA ligase. In another embodiment, PreCR® Repair Mix (available from New England Biolabs, Ipswich, Mass.) is used to repair the damaged DNA prior to subsequent processing steps.

At step 1420 double-strand DNA adapters are ligated to the dsDNA molecules obtained from step 1415 in a ligation reaction to generate a plurality of dsDNA adapter-molecule constructs. The ligation reaction can be performed using any suitable ligation step (e.g., using a ligase) which joins the dsDNA adapters to the dsDNA molecules to form dsDNA adapter-molecule constructs. In one example, the ligation reaction is performed using T4 DNA ligase. In another example, T7 DNA ligase is used for adapter ligation to the modified nucleic acid molecule. As described above, the ends of the dsDNA molecules may be repaired, phosphorylated and/or end-tailed prior to ligation of adapters to the ends of the dsDNA molecules.

As noted above, the dsDNA adapters may comprise a unique molecular identifier (UMI) sequence. The unique sequence tag can comprise a short oligonucleotide sequence having a length of from about 2 nt to about 100 nt, from about 2 nt to about 60 nt, from about 2 to about 40 nt, or from about 2 to about 20 nt. In another embodiment, the UMI tag may comprise a short oligonucleotide sequence greater than about 5, 6, 7, 8, 9, 10, 11, 12, 13, 14, 15, 16, 17, or 18 nucleotides (nt) in length. Also like the ssDNA adapters described above, the dsDNA adapters may include a universal primer and/or one or more sequencing oligonucleotides for use in subsequent cluster generation and/or sequencing (e.g., known P5 and P7 sequences for used in sequencing by synthesis (SBS) (Illumina, San Diego, Calif.)).

At step 1425 a portion of the sequence library is sequenced to obtain sequencing data or sequence reads, and the sequencing data or sequence reads analyzed. In general, any method known in the art can be used to obtain sequence data or sequence reads from a test sample. For example, in one embodiment, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step is performed prior to sequencing. In certain embodiments, the sequencing comprises whole genome sequencing (or shotgun sequencing) of the cfDNA library to provide sequence data or sequencing reads representative of a whole genome. In other embodiments, the sequencing comprises targeted sequencing of the cfDNA library. For example, the sequencing library can be enriched for specific target sequences (e.g., using a plurality of hybridization probes to pull down cfDNA fragments known to be, or suspected of being, indicative of cancer) and the targeted sequences sequenced.

At step 1430, the sequencing data or sequence reads can be analyzed for detecting the presence of absence of cancer, screening for cancer, determining cancer stage or status, monitoring cancer progression, and/or for determining a cancer classification (e.g., cancer type or cancer tissue of origin). In another embodiment, the sequencing data or sequence reads can be used to infer the presence or absence of cancer, cancer status and/or a cancer classification. For example, the sequencing data or sequencing reads can be analyzed to identify one or more mutational signatures indicative of cancer (see, e.g., U.S. Patent Application No. 62/469,984, filed Mar. 10, 2017). Alternatively, machine learning can be used for the detection and/or classification of cancer based on one or more parameters determined from sequencing data or sequencing reads (see, e.g., U.S. Patent Application No. 62/553,670, filed Sep. 1, 2017).

In one embodiment, the sequencing data or sequence reads can be analyzed to detect the presence or absence of, screening for, determine the stage or status of, monitor progression of, and/or classify a carcinoma, a sarcoma, a myeloma, a leukemia, a lymphoma, a blastoma, a germ cell tumor, or any combination thereof. In some embodiments, the carcinoma may be an adenocarcinoma. In other embodiments, the carcinoma may be a squamous cell carcinoma. In still other embodiments, the carcinoma is selected from the group consisting of: small cell lung cancer, non-small-cell lung, nasopharyngeal, colorectal, anal, liver, urinary bladder, cervical, testicular, ovarian, gastric, esophageal, head-and-neck, pancreatic, prostate, renal, thyroid, melanoma, and breast carcinoma. In another embodiment, the sequencing data or sequence reads can be analyzed to detect presence or absence of, screening for, determine the stage or status of, monitor progression of, and/or classify a sarcoma. In certain embodiments, the sarcoma can be selected from the group consisting of: osteosarcoma, chondrosarcoma, leiomyosarcoma, rhabdomyosarcoma, mesothelial sarcoma (mesothelioma), fibrosarcoma, angiosarcoma, liposarcoma, glioma, and astrocytoma. In still another embodiment, the sequencing data or sequence reads can be analyzed to detect presence or absence of, screening for, determine the stage or status of, monitor progression of, and/or classify leukemia. In certain embodiments, the leukemia can be selected from the group consisting of: myelogenous, granulocytic, lymphatic, lymphocytic, and lymphoblastic leukemia. In still another embodiment, the sequencing data or sequence reads can be used to detect presence or absence of, screening for, determine the stage or status of, monitor progression of, and/or classify a lymphoma. In certain embodiments, the lymphoma can be selected from the group consisting of: Hodgkin's lymphoma and Non-Hodgkin's lymphoma.

Aspects of the invention include methods that facilitate enhanced recovery of sequenceable double-stranded DNA (dsDNA) molecules, as well as a reduction in error rates associated with a sequencing library preparation procedure utilizing both strands of the dsDNA molecules (i.e., duplex error correction (see, e.g., U.S. Patent Appl. No. 2015/0024950, which is incorporated herein by reference). Methods in accordance with embodiments of the invention can employ, for example, a combination approach involving one or more pretreatment steps, followed by a sequencing library preparation procedure. In one embodiment, a method involves a DNA template repair pretreatment step that employs an enzyme cocktail that is formulated to repair damaged template DNA prior to its use in a PCR reaction. One non-limiting example of a DNA template repair pretreatment step enzyme cocktail is a PreCR® repair mix (New England Biolabs). In some embodiments, a method involves pretreatment with an exonuclease enzyme that cleaves single-stranded DNA ends in the 5′ and the 3′ direction, but is not active on linear or circularized double-stranded DNA. One non-limiting example of an exonuclease enzyme that can be utilized in connection with the present methods is Exonuclease VII (Exo VII; Exo7) (New England Biolabs). In some embodiments, the exonuclease is a single strand DNA exonuclease that does not cleave double stranded DNA. In some embodiments, optionally, an SPRI cleanup procedure is used after a pretreatment step, but before a library preparation step. In some embodiments, a heat inactivation step can be used to deactivate the exonuclease enzyme. The heat inactivation step may include heat-based deactivation of an exonuclease enzyme, for example, the reaction mixture can be heated to a temperature that ranges from about 50° C. to about 95° C., or from about 60° C. to about 80° C., or at about 70° C., for a period of time that ranges from about 5 min to about 2 hours, from about 10 min to about 1 hour, or from about 20 min to about 40 min. In certain embodiments, an SPRI cleanup procedure is not necessary when a heat inactivation step is utilized, and can optionally be omitted. In some embodiments, the methods employ an end repair (ER) procedure prior to library preparation.

In certain embodiments, a method can employ a combination approach wherein two or more pretreatments are utilized. For example, in some embodiments, a method involves preforming an exonuclease pretreatment step to remove ssDNA, and also employs a DNA template repair pretreatment step prior to performing a sequencing library preparation procedure. Details relating to a non-limiting example of a combination approach are provided in Example 1. In some embodiments, an SPRI cleanup procedure is used after each individual pretreatment step utilized in the combination approach, e.g., after the exonuclease pretreatment step, as well as after the DNA template repair pretreatment step, but before the sequencing library preparation procedure. In some embodiments, the methods employ an end repair (ER) procedure prior to library preparation.

Methods that employ one or more pretreatment steps, or a combination of two or more pretreatment steps, can be used to achieve a reduction in error rates associated with sequencing library preparation. For example, in some embodiments, incorporation of one or more pretreatment steps, or a combination of two or more pretreatment steps, results in an error rate reduction that ranges from about 40% to about 95%, such as about 45%, 50%, 55%, 60%, 65%, 70%, 75%, 80%, 85%, or about 90%. In other embodiments, incorporation of one or more pretreatment steps, or a combination of two of more pretreatment steps, results in an error rate reduction of at least 50%, at least 60%, at least 70%, at least 80%, or at least 90%.

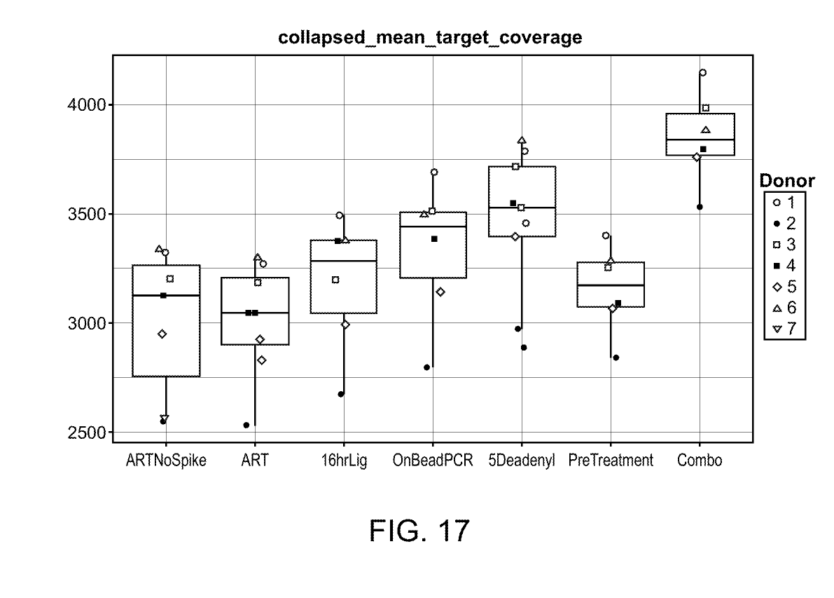

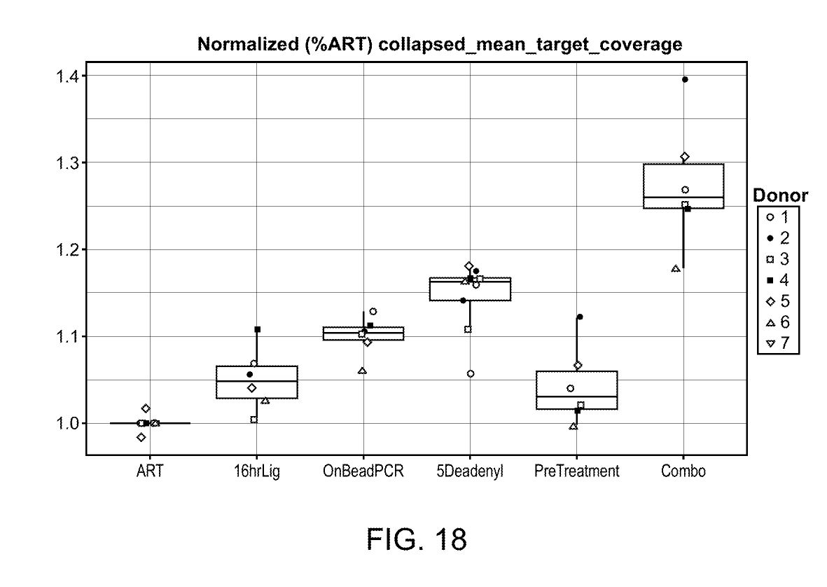

Aspects of the invention include modified sequencing library preparation reaction mixtures and conditions that can result in improvements to mean target coverage. For example, in some embodiments, the present methods involve longer ligation times, in some embodiments ranging from about 4 hour to about 20 hours, such as about 5, 6, 7, 8, 9, 10, 11, 12, 13, 14, 15, 16, 17, 18, or about 19 hours. In some embodiments, the methods involve on-bead PCR protocols. In some embodiments, the methods involve the incorporation of a 5′-deadenylase into the reaction mixture. As depicted in FIG. 17, an increased ligation time of 16 hours (increased from 4 hours) resulted in an increase in mean target coverage. On-bead PCR and incorporation of 5′-deadenylase into the reaction mixture also resulted in an increase in mean target coverage. The greatest increase in mean target coverage was achieved with a combination of increased ligation time, on-bead PCR, 5′-deadenylase, and pretreatment. These findings are also demonstrated in FIG. 18, which shows target coverage normalized to the control reaction conditions.

### Sequencing and Bioinformatics

As reviewed above, aspects of the invention include sequencing of nucleic acid molecules to generate a plurality of sequence reads, compilation of a plurality of sequence reads into a sequencing library, and bioinformatic manipulation of the sequence reads and/or sequencing library to determine sequence information from a test sample (e.g., a biological sample). In some embodiments, one or more aspects of the subject methods are conducted using a suitably-programmed computer system, as described further herein.

In certain embodiments, a sample is collected from a subject, followed by enrichment for genetic regions or genetic fragments of interest. For example, in some embodiments, a sample can be enriched by hybridization to a nucleotide array comprising cancer-related genes or gene fragments of interest. In some embodiments, a sample can be enriched for genes of interest (e.g., cancer-associated genes) using other methods known in the art, such as hybrid capture. See, e.g., Lapidus (U.S. Pat. No. 7,666,593), the contents of which is incorporated by reference herein in its entirety. In one hybrid capture method, a solution-based hybridization method is used that includes the use of biotinylated oligonucleotides and streptavidin coated magnetic beads. See, e.g., Duncavage et al., J Mol Diagn. 13(3): 325-333 (2011); and Newman et al., Nat Med. 20(5): 548-554 (2014). Isolation of nucleic acid from a sample in accordance with the methods of the invention can be done according to any method known in the art.

Sequencing may be by any method or combination of methods known in the art. For example, known DNA sequencing techniques include, but are not limited to, classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, Polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977), the contents of which are incorporated by reference herein in their entirety. Another conventional sequencing method involves chemical degradation of nucleic acid fragments. See, Maxam et al., Proc. Natl. Acad. Sci., 74: 560 564 (1977), the contents of which are incorporated by reference herein in their entirety. Methods have also been developed based upon sequencing by hybridization. See, e.g., Harris et al., (U.S. patent application number 2009/0156412), the contents of which are incorporated by reference herein in their entirety.

A sequencing technique that can be used in the methods of the provided invention includes, for example, Helicos True Single Molecule Sequencing (tSMS) (Harris T. D. et al. (2008) Science 320:106-109), the contents of which are incorporated by reference herein in their entirety. Further description of tSMS is shown, for example, in Lapidus et al. (U.S. Pat. No. 7,169,560), the contents of which are incorporated by reference herein in their entirety, Lapidus et al. (U.S. patent application publication number 2009/0191565, the contents of which are incorporated by reference herein in their entirety), Quake et al. (U.S. Pat. No. 6,818,395, the contents of which are incorporated by reference herein in their entirety), Harris (U.S. Pat. No. 7,282,337, the contents of which are incorporated by reference herein in their entirety), Quake et al. (U.S. patent application publication number 2002/0164629, the contents of which are incorporated by reference herein in their entirety), and Braslavsky, et al., PNAS (USA), 100: 3960-3964 (2003), the contents of which are incorporated by reference herein in their entirety.

Another example of a DNA sequencing technique that can be used in the methods of the provided invention is 454 sequencing (Roche) (Margulies, M et al. 2005, Nature, 437, 376-380, the contents of which are incorporated by reference herein in their entirety). Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application publication numbers 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the contents of each of which are incorporated by reference herein in their entirety).

In some embodiments, the sequencing technology is Illumina sequencing. Illumina sequencing is based on the amplification of DNA on a solid surface using fold-back PCR and anchored primers. Genomic DNA can be fragmented, or in the case of cfDNA, fragmentation is not needed due to the already short fragments. Adapters are ligated to the 5′- and 3′-ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. Yet another example of a sequencing technique that can be used in the methods of the provided invention is nanopore sequencing (Soni G V and Meller A. (2007) Clin Chem 53: 1996-2001, the contents of which are incorporated by reference herein in their entirety). Another example of a sequencing technique that can be used in the methods of the provided invention involves using a chemical-sensitive field effect transistor (chemFET) array to sequence DNA (for example, as described in US Patent Application Publication No. 20090026082, the contents of which are incorporated by reference herein in their entirety). Another example of a sequencing technique that can be used in the methods of the provided invention involves using an electron microscope (Moudrianakis E. N. and Beer M. Proc Natl Acad Sci USA. 1965 March; 53:564-71, the contents of which are incorporated by reference herein in their entirety).

If the nucleic acid from the sample is degraded or only a minimal amount of nucleic acid can be obtained from the sample, PCR can be performed on the nucleic acid in order to obtain a sufficient amount of nucleic acid for sequencing (See, e.g., Mullis et al. U.S. Pat. No. 4,683,195, the contents of which are incorporated by reference herein in its entirety).

### Biological Samples

Aspects of the invention involve obtaining a test sample, e.g., a biological sample, such as a tissue and/or body fluid sample, from a subject for purposes of analyzing a plurality of nucleic acids (e.g., a plurality of RNA molecules) therein. Samples in accordance with embodiments of the invention can be collected in any clinically-acceptable manner. Any test sample suspected of containing a plurality of nucleic acids can be used in conjunction with the methods of the present invention. In some embodiments, a test sample can comprise a tissue, a body fluid, or a combination thereof. In some embodiments, a biological sample is collected from a healthy subject. In some embodiments, a biological sample is collected from a subject who is known to have a particular disease or disorder (e.g., a particular cancer or tumor). In some embodiments, a biological sample is collected from a subject who is suspected of having a particular disease or disorder.

As used herein, the term “tissue” refers to a mass of connected cells and/or extracellular matrix material(s). Non-limiting examples of tissues that are commonly used in conjunction with the present methods include skin, hair, finger nails, endometrial tissue, nasal passage tissue, central nervous system (CNS) tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or non-human mammal. Tissue samples in accordance with embodiments of the invention can be prepared and provided in the form of any tissue sample types known in the art, such as, for example and without limitation, formalin-fixed paraffin-embedded (FFPE), fresh, and fresh frozen (FF) tissue samples.

As used herein, the term “body fluid” refers to a liquid material derived from a subject, e.g., a human or non-human mammal. Non-limiting examples of body fluids that are commonly used in conjunction with the present methods include mucous, blood, plasma, serum, serum derivatives, synovial fluid, lymphatic fluid, bile, phlegm, saliva, sweat, tears, sputum, amniotic fluid, menstrual fluid, vaginal fluid, semen, urine, cerebrospinal fluid (CSF), such as lumbar or ventricular CSF, gastric fluid, a liquid sample comprising one or more material(s) derived from a nasal, throat, or buccal swab, a liquid sample comprising one or more materials derived from a lavage procedure, such as a peritoneal, gastric, thoracic, or ductal lavage procedure, and the like.

In some embodiments, a test sample can comprise a fine needle aspirate or biopsied tissue. In some embodiments, a test sample can comprise media containing cells or biological material. In some embodiments, a test sample can comprise a blood clot, for example, a blood clot that has been obtained from whole blood after the serum has been removed. In some embodiments, a test sample can comprise stool. In one preferred embodiment, a test sample is drawn whole blood. In one aspect, only a portion of a whole blood sample is used, such as plasma, red blood cells, white blood cells, and platelets. In some embodiments, a test sample is separated into two or more component parts in conjunction with the present methods. For example, in some embodiments, a whole blood sample is separated into plasma, red blood cell, white blood cell, and platelet components.

In some embodiments, a test sample includes a plurality of nucleic acids not only from the subject from which the test sample was taken, but also from one or more other organisms, such as viral DNA/RNA that is present within the subject at the time of sampling.

Nucleic acid can be extracted from a test sample according to any suitable methods known in the art, and the extracted nucleic acid can be utilized in conjunction with the methods described herein. See, e.g., Maniatis, et al., Molecular Cloning: A Laboratory Manual, Cold Spring Harbor, N.Y., pp. 280-281, 1982, the contents of which are incorporated by reference herein in their entirety.

In one preferred embodiment, cell free nucleic acid (e.g., cell-free DNA (cfDNA) and/or cell-free RNA (cfRNA)) are extracted from a test sample. cfDNA are short base nuclear-derived DNA fragments present in several bodily fluids (e.g. plasma, stool, urine). See, e.g., Mouliere and Rosenfeld, PNAS 112(11): 3178-3179 (March 2015); Jiang et al., PNAS (March 2015); and Mouliere et al., Mol Oncol, 8(5):927-41 (2014). Tumor-derived circulating tumor nucleic acids (e.g., ctDNA and/or ctRNA) constitutes a minority population of cfNAs (i.e., cfDNA and/or cfRNA), in some cases, varying up to about 50%. In some embodiments, ctDNA and/or ctRNA varies depending on tumor stage and tumor type. In some embodiments, ctDNA and/or ctRNA varies from about 0.001% up to about 30%, such as about 0.01% up to about 20%, such as about 0.01% up to about 10%. The covariates of ctDNA and/or ctRNA are not fully understood, but appear to be positively correlated with tumor type, tumor size, and tumor stage. E.g., Bettegowda et al, Sci Trans Med, 2014; Newmann et al, Nat Med, 2014. Despite the challenges associated with the low population of ctDNA/ctRNA in cfNAs, tumor variants have been identified in ctDNA and/or ctRNA across a wide span of cancers. E.g., Bettegowda et al, Sci Trans Med, 2014. Furthermore, analysis of cfDNA and/or cfRNA versus tumor biopsy is less invasive, and methods for analyzing, such as sequencing, enable the identification of sub-clonal heterogeneity. Analysis of cfDNA and/or cfRNA has also been shown to provide for more uniform genome-wide sequencing coverage as compared to tumor tissue biopsies. In some embodiments, a plurality of cfDNA and/or cfRNA are extracted from a sample in a manner that reduces or eliminates co-mingling of cfDNA and genomic DNA. For example, in some embodiments, a sample is processed to isolate a plurality of the cfDNA and/or cfRNA therein in less than about 2 hours, such as less than about 1.5, 1 or 0.5 hours.

A non-limiting example of a procedure for preparing nucleic acid from a blood sample follows. Blood may be collected in 10 mL EDTA tubes (for example, the BD VACUTAINER® family of products from Becton Dickinson, Franklin Lakes, N.J.), or in collection tubes that are adapted for isolation of cfDNA (for example, the CELL FREE DNA BCT® family of products from Streck, Inc., Omaha, Nebr.) can be used to minimize contamination through chemical fixation of nucleated cells, but little contamination from genomic DNA is observed when samples are processed within 2 hours or less, as is the case in some embodiments of the present methods. Beginning with a blood sample, plasma may be extracted by centrifugation, e.g., at 3000 rpm for 10 minutes at room temperature minus brake. Plasma may then be transferred to 1.5 ml tubes in 1 ml aliquots and centrifuged again at 7000 rpm for 10 minutes at room temperature. Supernatants can then be transferred to new 1.5 ml tubes. At this stage, samples can be stored at −80° C. In certain embodiments, samples can be stored at the plasma stage for later processing, as plasma may be more stable than storing extracted cfDNA and/or cfRNA.

Plasma DNA and/or RNA can be extracted using any suitable technique. For example, in some embodiments, plasma DNA and/or RNA can be extracted using one or more commercially available assays, for example, the QIAmp Circulating Nucleic Acid Kit family of products (Qiagen N.V., Venlo Netherlands). In certain embodiments, the following modified elution strategy may be used. DNA and/or RNA may be extracted using, e.g., a QIAmp Circulating Nucleic Acid Kit, following the manufacturer's instructions (maximum amount of plasma allowed per column is 5 mL). If cfDNA and/or cfRNA are being extracted from plasma where the blood was collected in Streck tubes, the reaction time with proteinase K may be doubled from 30 min to 60 min. Preferably, as large a volume as possible should be used (i.e., 5 mL). In various embodiments, a two-step elution may be used to maximize cfDNA and/or cfRNA yield. First, DNA and/or RNA can be eluted using 30 μL of buffer AVE for each column. A minimal amount of buffer necessary to completely cover the membrane can be used in the elution in order to increase cfDNA and/or cfRNA concentration. By decreasing dilution with a small amount of buffer, downstream desiccation of samples can be avoided to prevent melting of double stranded DNA or material loss. Subsequently, about 30 μL of buffer for each column can be eluted. In some embodiments, a second elution may be used to increase DNA and/or RNA yield.

In other embodiments, RNA can be extracted and/or isolated using any suitable technique. For example, in some embodiments, RNA can be extracted using a commercially-available kit and/or protocol, e.g., a QIAamp Circulating Nucleic Acids kit and micro RNA extraction protocol.

In some embodiments, the methods involve DNase treating an extracted nucleic acid sample to remove cell-free DNA from a mixed cfDNA and cfRNA test sample.

### Computer Systems and Devices

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processors of any kind of digital computer. Generally, a processor will receive instructions and data from a read-only memory or a random access memory, or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. For example, a reference set of data may be stored at a remote location and a computer can communicate across a network to access the reference data set for comparison purposes. In other embodiments, however, a reference data set can be stored locally within the computer, and the computer accesses the reference data set within the CPU for comparison purposes. Examples of communication networks include, but are not limited to, cell networks (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, a data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium that can be used to store the desired information, and which can be accessed by a computing device.

Functions described herein can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the invention, a computer system for implementing some or all of the described inventive methods can include one or more processors (e.g., a central processing unit (CPU) a graphics processing unit (GPU), or both), main memory and static memory, which communicate with each other via a bus.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to, one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the invention can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database included in a system. Any suitable database structure may be used including relational databases, object-oriented databases, and others. In some embodiments, reference data is stored in a relational database such as a “not-only SQL” (NoSQL) database. In certain embodiments, a graph database is included within systems of the invention. It is also to be understood that the term “database” as used herein is not limited to one single database; rather, multiple databases can be included in a system. For example, a database can include two, three, four, five, six, seven, eight, nine, ten, fifteen, twenty, or more individual databases, including any integer of databases therein, in accordance with embodiments of the invention. For example, one database can contain public reference data, a second database can contain test data from a patient, a third database can contain data from healthy individuals, and a fourth database can contain data from sick individuals with a known condition or disorder. It is to be understood that any other configuration of databases with respect to the data contained therein is also contemplated by the methods described herein.

References and citations to other documents, such as patents, patent applications, patent publications, journals, books, papers, web contents, have been made throughout this disclosure. All such documents are hereby incorporated herein by reference in their entirety for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof. All references cited throughout the specification are expressly incorporated by reference herein.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

### EXAMPLES

### Example 1: Reduction in Error Rate Resulting from Pretreatment Procedures

Sequencing libraries were generated using 7 different protocols to determine error reduction rates attributable to different combinations of pretreatment procedures. Using 30 ng of cfDNA as the input material, 7 different protocols (each employing different combinations of pretreatment steps) were carried out. The first protocol was a known library preparation method utilizing steps for end repair, A-tailing, adapter ligation, SPRI cleanup and PCR amplification (control). The second protocol incorporated a PreCR pretreatment step, followed by an SPRI cleanup. The third protocol incorporated a PreCR pretreatment step, with no SPRI cleanup. The fourth protocol incorporated an Exo7 pretreatment step, followed by an SPRI cleanup. The fifth protocol incorporated an Exo7 pretreatment step, with no SPRI cleanup, but with a heat inactivation step instead (40 min at 70° C.), as described above. The sixth protocol incorporated an Exo7 pretreatment step, followed by a PreCR pretreatment step, followed by an end repair (ER) step. The seventh protocol incorporated an Exo7 pretreatment step, followed by an SPRI cleanup step, followed by a PreCR pretreatment step, followed by an SPRI cleanup step, followed by an end repair (ER) step.

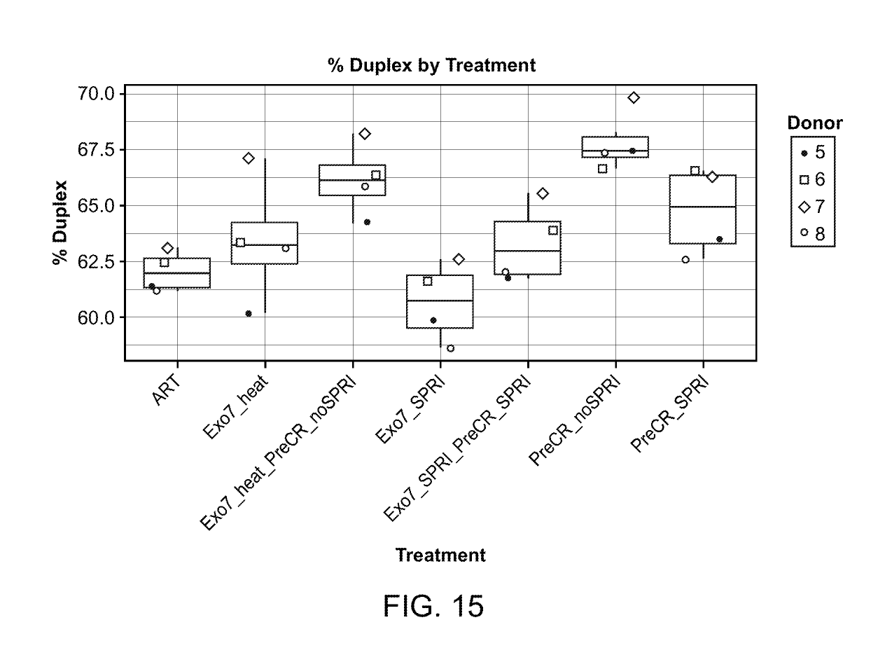

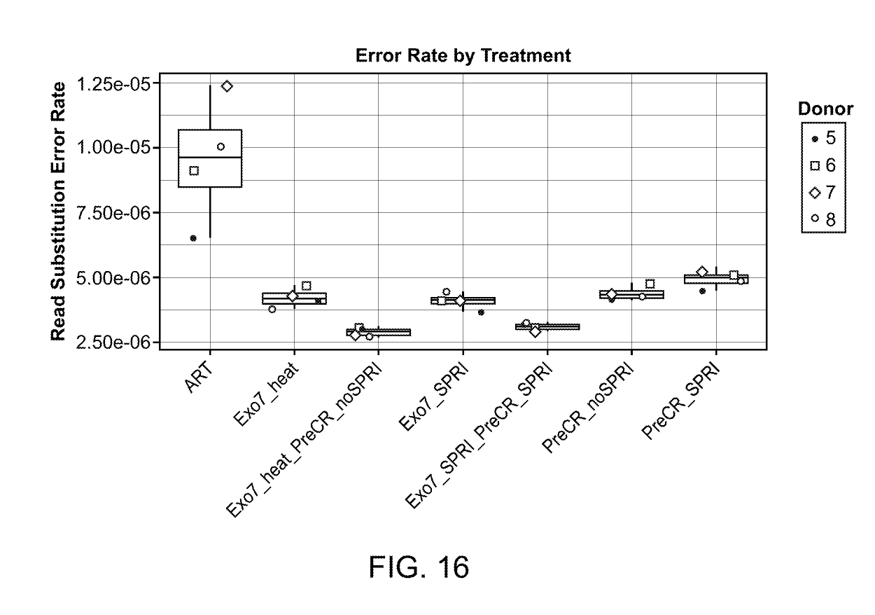

The results are provided in FIGS. 15-16, which show recovery of duplex DNA strands (as a percentage of all nucleic acids recovered) and read substitution error rate as a function of the preparation protocol used to generate each sequence library.

The control protocol resulted in an average percentage of duplex DNA of approximately 62%. The Exo7 plus heat kill protocol increased the average percentage of duplex DNA to approximately 63%. The Exo7 plus heat kill plus PreCR with no SPRI protocol increased the average percentage of duplex DNA to approximately 66%. The Exo7 plus SPRI protocol resulted in a lower average percentage of duplex DNA than the control protocol, approximately 61%. The Exo7 plus SPRI plus PreCR plus SPRI protocol resulted in an average percentage of duplex DNA of approximately 63%, and was comparable to the Exo7 plus heat kill protocol. The PreCR with no SPRI protocol resulted in the highest average percentage of duplex DNA, approximately 67.5%. The PreCR plus SPRI protocol resulted in a slightly lower average percentage of duplex DNA, approximately 65%.

The control protocol resulted in an average error rate of approximately 9×10⁻⁶. In contrast, all of the other preparation protocols significantly reduced the observed error rate. The lowest error rate (approximately 3×10⁻⁶) was observed from the protocols that employed both the Exo7 and PreCR pretreatment steps.

1. A method for preparing a sequencing library from a test sample comprising a plurality of double-stranded DNA (dsDNA) molecules, the method comprising:
(a) obtaining a test sample comprising a plurality of dsDNA molecules, wherein the dsDNA molecules comprise one or more free single-stranded DNA (ssDNA) overhangs at one or both ends of the dsDNA molecules;
(b) treating the dsDNA molecules to remove the free ssDNA overhangs, thereby generating a plurality of blunt ended dsDNA molecules;
(c) modifying the blunt ended dsDNA molecules for adapter ligation;
(d) ligating a plurality of dsDNA adapters to the plurality of blunt ended dsDNA molecules obtained from step (c) to generate a plurality of dsDNA adapter-molecule constructs; and
(e) amplifying the dsDNA adapter-molecule constructs to generate a sequencing library.

2. The method according to claim 1, wherein treating the dsDNA molecules to remove the free ssDNA overhangs comprises an exonuclease pretreatment step, a DNA template repair pretreatment step, a heat inactivation step, or a combination thereof.

3. The method according to claim 1, further comprising:
(f) sequencing the sequencing library to obtain a plurality of sequence reads; and
(g) detecting the presence or absence of cancer, determining cancer status, monitoring cancer progression and/or determining a cancer classification from the plurality of sequence reads.

4. The method according to claim 1, wherein the dsDNA molecules are cell-free DNA (cfDNA) fragments.

5. The method according to claim 4, wherein the cfDNA fragments originate from healthy cells and from cancer cells.

6. (canceled)

7. The method according to claim 1, wherein the free single-stranded overhang comprises a free 5-end.

8. The method according to claim 1, wherein the free single-stranded DNA overhang comprises a free 3′-end.

9. The method according to claim 2, wherein the exonuclease pretreatment step comprises a single strand DNA nuclease.

10. (canceled)

11. (canceled)

12. The method according to claim 9, wherein removal of the free single-stranded DNA using the single-strand DNA nuclease results in a plurality of blunt ended dsDNA molecules.

13. The method according to claim 1, wherein modification of the plurality of dsDNA fragments comprises end-repairing and A-tailing prior to ligation step (d).

14. The method according to claim 1, wherein the adapters further comprise a sample-specific index sequence.

15. The method according to claim 1, wherein the adapters further comprise a universal priming site.

16. The method according to claim 1, wherein the adapters further comprise one or more sequencing oligonucleotides for use in cluster generation and/or sequencing.

17. (canceled)

18. (canceled)

19. (canceled)

20. The method according to claim 3, wherein monitoring cancer progression further comprises monitoring disease progression, monitoring therapy, or monitoring cancer growth.

21. The method according to claim 3, wherein the cancer classification further comprises determining a cancer type and/or a cancer tissue of origin.

22. The method according to claim 3, wherein monitoring cancer progression further comprises monitoring disease progression, monitoring therapy, or monitoring cancer growth.

23. (canceled)

24. A method for preparing a sequencing library from a test sample comprising a plurality of double-stranded DNA (dsDNA) molecules, the method comprising:
(a) obtaining a test sample comprising a plurality of dsDNA molecules;
(b) treating the dsDNA molecules to remove and/or repair one or more uracil residues within the dsDNA molecules;
(c) modifying the plurality of dsDNA fragments for adapter ligation;
(d) ligating a plurality of dsDNA adapters to the plurality of dsDNA molecules obtained from step (c) to generate a plurality of dsDNA adapter-molecule constructs; and
(e) amplifying the dsDNA adapter-molecule constructs to generate a sequencing library.

25. The method according to claim 24, further comprising:
(f) sequencing the sequencing library to obtain a plurality of sequence reads; and
(g) detecting the presence or absence of cancer, determining cancer status, monitoring cancer progression and/or determining a cancer classification from the plurality of sequence reads.

26. (canceled)

27. (canceled)

28. (canceled)

29. The method according to claim 24, wherein a uracil-specific excision reagent is used to remove one or more uracil residues from the dsDNA molecules.

30. The method according to claim 29, wherein the removed uracil residue is replaced with a cytosine residue using a DNA polymerase and/or a DNA ligase.

31.-49. (canceled)
